(19) United States
(12) Patent Application Publication
(10) Pub. No.: US 2025/0296998 A1
(43) Pub. Date: Sep. 25, 2025

(54) Methods of Treating Crohn's Disease with Anti-IL23 Specific Antibody

(71) Applicant: Janssen Biotech, Inc., 19044, PA (US)

(72) Inventors: Omoniyi Adedokun, Phoenixville, PA (US); Daphne Chan, Abington, PA (US); Yang Chen, Ambler, PA (US); Philippe Szapary, Wynnewood, PA (US); Jewel Johanns, Devon, PA (US); Zijiang Yang, Ambler, PA (US); Natalie A Terry, Philadelphia, PA (US)

(21) Appl. No.: 19/084,954

(22) Filed: Mar. 20, 2025

Related U.S. Application Data
(60) Provisional application No. 63/567,545, filed on Mar. 20, 2024.

(51) Int. Cl. C07K 16/24

(52) U.S. Cl. CPC C07K 16/244; C07K 2317/24

(57) ABSTRACT

A method of treating Crohn's disease in a patient administers an IL-23 specific antibody, e.g., guselkumab, at an initial intravenous dose and subsequent subcutaneous doses in order for the patient to respond to the antibody and meet one or more of the clinical endpoints.

### CROSS-REFERENCE TO RELATED APPLICATIONS

This application claims priority to U.S. Provisional Patent Application No. 63/567,545, filed on Mar. 20, 2024, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an xml formatted sequence listing with a file name “JBI6893USNP1 Sequence Listing” and a creation date of Mar. 18, 2025, and having a size of 11 kb. The sequence listing submitted via USPTO Patent Center is part of the specification and is herein incorporated by reference in its entirety.

### FIELD OF THE INVENTION

The present invention concerns methods for treating Crohn's Disease with an antibody that binds the human IL-23 protein. In particular, it relates to dosing regimens for administration of an anti-IL-23 specific antibody and specific pharmaceutical compositions of an antibody, e.g., guselkumab.

### BACKGROUND OF THE INVENTION

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rβ1) chain binds to the p40 subunit of IL-12, providing the primary interaction between IL-12 and its receptor. However, it is IL-12p35 ligation of the second receptor chain, IL-12Rβ2, that confers intracellular signaling (e.g. STAT4 phosphorylation) and activation of the receptor-bearing cell (Presky et al, 1996). IL-12 signaling concurrent with antigen presentation is thought to invoke T cell differentiation towards the T helper 1 (Th1) phenotype, characterized by interferon gamma (IFNγ) production (Trinchieri, 2003). Th1 cells are believed to promote immunity to some intracellular pathogens, generate complement-fixing antibody isotypes, and contribute to tumor immunosurveillance. Thus, IL-12 is thought to be a significant component to host defense immune mechanisms.

It was discovered that the p40 protein subunit of IL-12 can also associate with a separate protein subunit, designated p19, to form a novel cytokine, IL-23 (Oppman et al, 2000). IL-23 also signals through a two-chain receptor complex. Since the p40 subunit is shared between IL-12 and IL-23, it follows that the IL-12Rβ1 chain is also shared between IL-12 and IL-23. However, it is the IL-23p19 ligation of the second component of the IL-23 receptor complex, IL-23R, that confers IL-23 specific intracellular signaling (e.g., STAT3 phosphorylation) and subsequent IL-17 production by T cells (Parham et al, 2002; Aggarwal et al. 2003). Recent studies have demonstrated that the biological functions of IL-23 are distinct from those of IL-12, despite the structural similarity between the two cytokines (Langrish et al, 2005).

Abnormal regulation of IL-12 and Th1 cell populations has been associated with many immune-mediated diseases since neutralization of IL-12 by antibodies is effective in treating animal models of psoriasis, multiple sclerosis (MS), rheumatoid arthritis, inflammatory bowel disease, insulin-dependent (type 1) diabetes mellitus, and uveitis (Leonard et al, 1995; Hong et al, 1999; Malfait et al, 1998; Davidson et al, 1998). However, since these studies targeted the shared p40 subunit, both IL-12 and IL-23 were neutralized in vivo. Therefore, it was unclear whether IL-12 or IL-23 was mediating disease, or if both cytokines needed to be inhibited to achieve disease suppression. Recent studies have confirmed through IL-23p19 deficient mice or specific antibody neutralization of IL-23 that IL-23 inhibition can provide equivalent benefit as anti-IL-12p40 strategies (Cua et al, 2003, Murphy et al, 2003, Benson et al 2004). Therefore, there is increasing evidence for the specific role of IL-23 in immune-mediated disease. Neutralization of IL-23 without inhibition of IL-12 pathways could then provide effective therapy of immune-mediated disease with limited impact on important host defense immune mechanism. This would represent a significant improvement over current therapeutic options.

Currently, there are three classes of biologic agents approved for the treatment of moderately to severely active Crohn's disease: tumor necrosis factor (TNF) antagonist therapies (infliximab, adalimumab, certolizumab), integrin inhibitors (natalizumab and vedolizumab), and an IL-12/23 inhibitor (ustekinumab). Although the introduction of biologic agents has significantly improved the clinical management of patients with moderately to severely active Crohn's disease, a sizable proportion of the target patient population is non-responsive or will lose response over time. A review of the available data for approved biologic agents highlighted the unmet need in achieving and maintaining long-term remission, especially among patients who have previously failed biologic treatments. In all-treated patients (ie, all patients who were randomized at Week 0 of the studies evaluated), the estimated rates of clinical remission at 1 year in the biologic failure or intolerance (BIO-Failure) population is around 20%, and ranges from 20% to 50% in the conventional therapy failure or intolerance (CON-Failure) population.

In summary, there remains considerable unmet medical need for new treatment options, especially therapies with novel mechanisms of action that have the potential to raise the efficacy bar and maximize the proportion of patients who achieve and maintain clinical remission.

### SUMMARY OF THE INVENTION

In a first aspect, the invention concerns a method of a subject suffering from Crohn's disease comprising administering an anti-IL-23 specific antibody (also referred to as IL-23p19 antibody), e.g., guselkumab, to the patient in an initial intravenous induction dose from the start of treatment until 8 weeks from the start of treatment, and then subcutaneously administering the anti-IL-23 specific antibody once every 4 or 8 weeks thereafter, e,g., a dose at 0, 4, 8, 12 or 16, 20 or 24, 28 or 32, 36 or 40, 44 or 48 weeks. In addition, in another embodiment the subcutaneous treatment continues through 140 weeks after the start of treatment.

In one embodiment, the subject receives the anti-IL-23 specific antibody at a dose of 1200, 600 or 200 mg intravenously initially, 4 weeks after the initial dose and 8 weeks after the initial dose and continue with subcutaneous treatment of the anti-IL-23 specific antibody at 100 or 200 mg every 4 weeks through 44 weeks after initial treatment.

In another aspect, the composition used in the method of the invention comprises a pharmaceutical composition comprising: an anti-IL-23 specific antibody. In a preferred embodiment, the anti-IL-23 specific antibody is guselkumab in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

In an embodiment, Crohn's disease patients achieved significant improvement in clinical endpoints selected from:
(i) Change from Baseline in the Crohn's Disease Activity lodex (CDAI) Score at Week 12 and/or Week 48. The CDAI score will be assessed by collecting information on 8 different Crohn's disease-related variables, with scores ranging from 0 to approximately 600. A decrease over time indicates improvement in disease activity.
(ii) Clinical remission at Week 12 and/or Week 48, defined as CDAI less than (<) 150 points.
(iii) Clinical response at Week 12 and/or Week 48, defined as greater than or equal to (>=) 100-point reduction from baseline in CDAI score or CDAI score <150.
(iv) Patient-Reported Outcome (PRO)-2 Remission at Week 12 and/or Week 48 defined based on average daily stool frequency (SF) and average daily abdominal pain (AP) score.
(v) Clinical Biomarker Response at Week 12 and/or Week 48 defined using clinical response based on the CDAI score and reduction from baseline in C-reactive protein (CRP) or fecal calprotectin.
(vi) Endoscopic response at Week 12 and/or Week 48 measured by the Simple Endoscopic Score for Crohn's Disease (SES-CD). The SES-CD is based on the evaluation of 4 endoscopic components across 5 ileocolonic segments, with a total score ranging from 0 to 56.
(vii) Endoscopic remission at Week 12 and/or Week 48 measured by the Simple Endoscopic Score for Crohn's Disease (SES-CD); SES-CD ≤2.
(viii) Clinical remission at Week 48 defined as CDAI score <150.
(ix) Durable Clinical Remission at Week 48 defined as CDAI <150 for most of all visits between Week 12 and Week 48.
(x) Corticosteroid-Free Clinical Remission at Week 48 defined as CDAI score <150 at Week 48 and not receiving corticosteroids at Week 48.
(xi) PRO-2 remission at Week 48 defined based on average daily stool frequency (SF) and average daily abdominal pain (AP) score. Fatigue response at Week 12 based on the Patient Reported Outcomes Measurement Information System (PROMIS). Fatigue Short Form 7a contains 7 items that evaluate the severity of fatigue, with higher scores indicating greater fatigue.
(xii) Deep remission at Week 48 measured by achieving both clinical remission and endoscopic remission.

In another aspect of the invention the pharmaceutical composition comprises an isolated anti-IL23 specific antibody having the guselkumab CDR sequences comprising (i) the heavy chain CDR amino acid sequences of SEQ ID NO: 1, SEQ ID NO: 2, and SEQ ID NO: 3; and (ii) the light chain CDR amino acid sequences of SEQ ID NO: 4, SEQ ID NO: 5, and SEQ ID NO: 6 in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

Another aspect of the method of the invention comprises administering a pharmaceutical composition comprising an isolated anti-IL-23 specific antibody having the guselkumab heavy chain variable region amino acid sequence of SEQ ID NO: 7 and the guselkumab light chain variable region amino acid sequence of SEQ ID NO: 8 in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

A father aspect of the method of the invention comprises administering a pharmaceutical composition comprising an isolated anti-IL-23 specific antibody having the guselkumab heavy chain amino acid sequence of SEQ ID NO: 9 and the guselkumab light chain amino acid sequence of SEQ ID NO: 10 in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

The details of one or more embodiments of the invention are set forth in the description below. Other features and advantages will be apparent from the following detailed description, figures, and the appended claims.

### BRIEF DESCRIPTION OF THE DRAWINGS

In the Figures:

FIG. 1A shows the composite endpoint of clinical response at Week 12 and clinical remission at Week 48 for Galaxi 2 and 3 trials of Guselkumab-treated patients versus placebo.

FIG. 1B shows the composite endpoint of clinical response at Week 12 and endoscopic response at Week 48 for Galaxi 2 and 3 trials of Guselkumab-treated patients versus placebo.

FIG. 1C shows various clinical endpoints comparing Guselkumab-treated patients to Ustekinumab-treated patients.

### DETAILED DESCRIPTION OF THE PREFERRED EMBODIMENTS

As used herein the method of treatment of a subject suffering from Crohn's disease comprises administering isolated, recombinant and/or synthetic anti-IL-23 specific human antibodies and diagnostic and therapeutic compositions, methods and devices.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CHI domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human or humanized, antibodies that have binding specificities for at least two different antigens. In the present case, one of the binding specificities is for at least one IL-23 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-IL-23 specific (also termed IL-23 specific antibodies) (or antibodies to IL-23) useful in the methods and compositions of the present invention can optionally be characterized by high affinity binding to IL-23 and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period.

The term “safe,” as it relates to a dose, dosage regimen, treatment or method with an anti-IL-23 antibody of the present invention (e.g., the anti-IL-23 antibody guselkumab), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator. An adverse event is an untoward medical occurrence in a patient administered a medicinal product. In particular, safe as it relates to a dose, dosage regimen or treatment with an anti-IL-23 antibody of the present invention refers to a relatively low or reduced frequency and/or low or reduced severity of adverse events associated with administration of the antibody if attribution is considered to be possible, probable, or very likely due to the use of the anti-IL-23 antibody.

### Utility

The isolated nucleic acids of the present invention can be used for production of at least one anti-IL-23 antibody or specified variant thereof, which can be used to measure or effect in a cell, tissue, organ or animal (including mammals and humans), to diagnose, monitor, modulate, treat, alleviate, help prevent the incidence of, or reduce the symptoms of Crohn's disease.

Such a method can comprise administering an effective amount of a composition or a pharmaceutical composition comprising at least one anti-IL-23 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### CITATIONS

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

### Antibodies of the Present Invention—Production and Generation

At least one anti-IL-23 antibody used in the method of the present invention can be optionally produced by a cell line, a mixed cell line, an immortalized cell or clonal population of immortalized cells, as well known in the art. Sec, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

A preferred anti-IL-23 antibody is guselkumab (also referred to as CNTO1959) having the heavy chain variable region amino acid sequence of SEQ ID NO: 7 and the light chain variable region amino acid sequence of SEQ ID NO: 8 and having the heavy chain CDR amino acid sequences of SEQ ID NO: 1, SEQ ID NO: 2, and SEQ ID NO: 3; and the light chain CDR amino acid sequences of SEQ ID NO: 4, SEQ ID NO: 5, and SEQ ID NO: 6. Other anti-IL-23 antibodies have sequences listed herein and are described in U.S. Pat. No. 7,935,344, the entire contents of which are incorporated herein by reference).

Human antibodies that are specific for human IL-23 proteins or fragments thereof can be raised against an appropriate immunogenic antigen, such as an isolated IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Sec, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, CA; Ixsys. Scc, e.g., EP 368,684, PCT/GB91/01134; PCT/GB92/01755; PCT/GB92/002240; PCT/GB92/00883; PCT/GB93/00605; U.S. Ser. No. 08/350,260 (5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins-U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, MA; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g., www.ncbi.nlm.nih.gov/entrez/query.fcgi; www.ncbi.nih.gov/igblast; www.atcc.org/phage/hdb.html; www.mrc-cpc.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research_tools.html; www.whfreeman.com/immunology/CH05/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wustl.edu/˜hcenter/index.html; www.appliedbiosystems.com; www.nal.usda.gov/awic/pubs/antibody; www.m.chime-u.ac.jp/˜yasuhito/Elisa.html; www.biodesign.com; www.cancerresearchuk.org; www.biotech.ufl.edu; www.isac-net.org; baserv.uci.kun.nl/˜jraats/links1.html; www.recab.uni-hd.de/immuno.bme.nwu.edu; www.mrc-cpe.cam.ac.uk; www.ibt.unam.mx/vir/V_mice.html; http://www.bioinf.org.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TAHHP.html; www.ibt.unam.mx/vir/structure/stat_aim.html; www.biosci.missouri.edu/smithgp/index.html; www.jerini.de; Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human IL-23 specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human IL-23 specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151:2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human IL-23 specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human IL-23 (or anti-IL-23) antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human IL-23 specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human IL-23 specific antibody of the present invention is expressed in cells that express beta (1,4)-N-acetylglucosaminyltransferase III (GnT III), such that GnT III adds GlcNAc to the human IL-23 antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The anti-IL-23 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-23 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6 (4) 579-591 (1994), Green et al, Nature Genetics 7:13-21 (1994), Mendez et al., Nature Genetics 15:146-156 (1997), Taylor et al., Nucleic Acids Research 20 (23): 6287-6295 (1992), Tuaillon et al., Proc Natl Acad Sci USA 90 (8) 3720-3724 (1993), Lonberg et al., Int Rev Immunol 13 (1): 65-93 (1995) and Fishwald et al., Nat Biotechnol 14 (7): 845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, CA), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL23 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL23 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. Sec, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-23 with a wide range of affinities (K_{D}). In a preferred embodiment, a human mAb can optionally bind human IL-23 with high affinity. For example, a human mAb can bind human IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

### Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one anti-IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-23 antibody or variable region; and nucleic acid molecules which comprise a nucleotide sequence substantially different from those described above but which, due to the degeneracy of the genetic code, still encode at least one anti-IL-23 antibody as described herein and/or as known in the art. Of course, the genetic code is well known in the art. Thus, it would be routine for one skilled in the art to generate such degenerate nucleic acid variants that code for specific anti-IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-23 antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

### Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

### Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

### Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (Scc, e.g., Ausubel, supra; or Sambrook, supra)

### Nucleic Acid Screening and Isolation Methods

A cDNA or genomic library can be screened using a probe based upon the sequence of a polynucleotide used in the method of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides used in the method of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, CA (1990). Commercially available kits for genomic PCR amplification are known in the art. Sec, e.g., Advantage-GC Genomic PCR Kit (Clontech). Additionally, e.g., the T4 gene 32 protein (Bochringer Mannheim) can be used to improve yield of long PCR products.

### Synthetic Methods for Constructing Nucleic Acids

The isolated nucleic acids used in the method of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

### Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1˜4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. Sec, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an Antibody

An anti-IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Anti-IL-23 Antibodies.

An anti-IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in the method of the present invention comprise the antibody amino acid sequences disclosed herein encoded by any suitable polynucleotide, or any isolated or prepared antibody. Preferably, the human antibody or antigen-binding fragment binds human IL-23 and, thereby, partially or substantially neutralizes at least one biological activity of the protein. An antibody, or specified portion or variant thereof, that partially or preferably substantially neutralizes at least one biological activity of at least one IL-23 protein or fragment can bind the protein or fragment and thereby inhibit activities mediated through the binding of IL-23 to the IL-23 receptor or through other IL-23-dependent or mediated mechanisms. As used herein, the term “neutralizing antibody” refers to an antibody that can inhibit an IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-23 antibody to inhibit an IL-23-dependent activity is preferably assessed by at least one suitable IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-IL-23 specific antibody can comprise at least one of a heavy or light chain variable region having a defined amino acid sequence. For example, in a preferred embodiment, the anti-IL-23 antibody comprises at least one of a heavy chain variable region, optionally having the amino acid sequence of SEQ ID NO:7 and/or at least one light chain variable region, optionally having the amino acid sequence of SEQ ID NO:8. In an additional preferred embodiment, the anti-IL-23 antibody comprises at least one heavy chain, optionally having the amino acid sequence of SEQ ID NO:9 and/or at least one light chain, optionally having the amino acid sequence of SEQ ID NO:10. Antibodies that bind to human IL-23 and that comprise a defined heavy or light chain variable region can be prepared using suitable methods, such as phage display (Katsube, Y., et al., Int J Mol. Med, 1 (5): 863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-23 with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine(S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Amino Acid Codes

The amino acids that make up anti-IL-23 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | THREE |
|---|---|---|---|
| LETTER | LETTER | | NUCLEOTIDE |
| CODE | CODE | NAME | CODON(S) |
| A | Ala | Alanine | GCA, GCC, GCG, |
| | | | GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, |
| | | | GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, |
| | | | CUC, CUG, CUU |
| M | Met | Methionine | AUG |
| N | Asn | Asparagine | AAC, AAU |
| P | Pro | Proline | CCA, CCC, CCG, |
| | | | CCU |
| Q | Gln | Glutamine | CAA, CAG |
| R | Arg | Arginine | AGA, AGG, CGA, |
| | | | CGC, CGG, CGU |
| S | Ser | Serine | AGC, AGU, UCA, |
| | | | UCC, UCG, UCU |
| T | Thr | Threonine | ACA, ACC, ACG, |
| | | | ACU |
| V | Val | Valine | GUA, GUC, GUG, |
| | | | GUU |
| W | Trp | Tryptophan | UGG |
| Y | Tyr | Tyrosine | UAC, UAU |

An anti-IL-23 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL-23 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-23 specific antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL-23 antibodies can include, but are not limited to, at least one portion, sequence or combination selected from 5 to all of the contiguous amino acids of at least one of SEQ ID NOS: 1, 2, 3, 4, 5, and 6.

IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-23 antibody can further optionally comprise a polypeptide of at least one of 70-100% of 5, 17, 10, 11, 7, 9, 119, or 108 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, MD).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12 (1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:

(1) Algorithm: Needleman and Wunsch, J. Mol Biol. 48:443-453 (1970) Comparison matrix: BLOSSUM62 from Hentikoff and Hentikoff, Proc. Natl. Acad. Sci, USA. 89:10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:

(1) Algorithm: Needleman and Wunsch, J. Mol Biol. 48:443-453 (1970)
Comparison matrix: matches=+10, mismatch=0
Gap Penalty: 50
Gap Length Penalty: 3

Available as: The “gap” program from Genetics Computer Group, Madison Wis. These are the default parameters for nucleic acid sequence comparisons.

By way of example, a polynucleotide sequence may be identical to another sequence, that is 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the sequence by the numerical percent of the respective percent identity (divided by 100) and subtracting that product from the total number of nucleotides in the sequence, or:
n.sub.n.ltorsim.x.sub.n-(x.sub.n.y),
wherein n.sub.n is the number of nucleotide alterations, x.sub.n is the total number of nucleotides in sequence, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the SEQ ID NOs above may create nonsense, missense or frameshift mutations in this coding sequence and thereby alter the polypeptide encoded by the polynucleotide following such alterations. Similarly, a polypeptide sequence may be identical to the reference sequence of the SEQ ID NOs above, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the SEQ ID NOs above by the numerical percent of the respective percent identity (divided by 100) and then subtracting that product from the total number of amino acids in the SEQ ID NOs above, or:
n.sub.a.ltorsim.x.sub.a-(x.sub.a.y),
wherein n.sub.a is the number of amino acid alterations, x.sub.a is the total number of amino acids in the SEQ ID NOs above, and y is, for instance 0.70 for 70%, 0.80 for 80%, 0.85 for 85% etc., and wherein any non-integer produce of x.sub.a and y is rounded down to the nearest integer prior to subtracting it from x.sub.a.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-23 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, CA (1996)). An activating group can be bonded directly to the organic group (e.g., hydrophilic polymer, fatty acid, fatty acid ester), or through a linker moiety, for example, a divalent C₁-C₁₂ group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnol. Bioeng., 56 (4): 456-463 (1997)), and the methods described in Hermanson, G. T., Bioconjugate Techniques, Academic Press: San Diego, CA (1996).

The method of the present invention also uses an anti-IL-23 antibody composition comprising at least one, at least two, at least three, at least four, at least five, at least six or more anti-IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-23 antibody amino acid sequence selected from the group consisting of 70-100% of the contiguous amino acids of the SEQ ID NOs above, or specified fragments, domains or variants thereof. Preferred anti-IL-23 antibody compositions include at least one or two full length, fragments, domains or variants as at least one CDR or LBP containing portions of the anti-IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

### Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, PA, 2001; Health Professional's Drug Guide 2001, ed., Shannon, Wilson, Stang, Prentice-Hall, Inc, Upper Saddle River, NJ; Pharmacotherapy Handbook, Wells et al., ed., Appleton & Lange, Stamford, CT, each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one corticosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (Sec, e.g., pp. 1098-1136 of Nursing 2001 Drug Handbook.)

Anti-IL-23 antibody compositions can further comprise at least one of any suitable and effective amount of a composition or pharmaceutical composition comprising at least one anti-IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-40 et al. (e.g., IL-1, IL-2, etc.). Suitable dosages are well known in the art. Sec, e.g., Wells et al., eds., Pharmacotherapy Handbook, 2nd Edition, Appleton and Lange, Stamford, CT (2000); PDR Pharmacopoeia, Tarascon Pocket Pharmacopoeia 2000, Deluxe Edition, Tarascon Publishing, Loma Linda, CA (2000), each of which references are entirely incorporated herein by reference.

Anti-IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, PA) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-23 antibody, portion or variant compositions according to the invention are known in the art, e.g., as listed in “Remington: The Science & Practice of Pharmacy,” 19th ed., Williams & Williams, (1995), and in the “Physician's Desk Reference,” 52^{nd} ed., Medical Economics, Montvale, NJ (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-23 specific antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-23 specific antibody, and a second vial comprising an aqueous diluent of prescribed buffer or preservative, wherein said packaging material comprises a label that instructs a patient to reconstitute the anti-IL-23 specific antibody in the aqueous diluent to form a solution that can be held over a period of twenty-four hours or greater.

The anti-IL-23 specific antibody used in accordance with the present invention can be produced by recombinant means, including from mammalian cell or transgenic preparations, or can be purified from other biological sources, as described herein or as known in the art.

The range of the anti-IL-23 specific antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-23 specific antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-23 specific antibody and preservative in an aqueous diluent is carried out using conventional dissolution and mixing procedures. To prepare a suitable formulation, for example, a measured amount of at least one anti-IL-23 specific antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®. NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, NJ, www.bectondickenson.com), Disetronic (Burgdorf, Switzerland, www.disetronic.com; Bioject, Portland, Oregon (www.bioject.com); National Medical Products, Weston Medical (Peterborough, UK, www.weston-medical.com), Medi-Ject Corp (Minneapolis, MN, www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products may include packaging material. The packaging material provides, in addition to the information required by the regulatory agencies, the conditions under which the product can be used. The packaging material of the present invention provides instructions to the patient, as applicable, to reconstitute the at least one anti-IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-23 specific mAbs bind to similar or different epitopes and/or compete with each other. Abs are individually coated on ELISA plates. Competing mAbs are added, followed by the addition of biotinylated hrIL-23. For positive control, the same mAb for coating may be used as the competing mAb (“self-competition”). IL-23 binding is detected using streptavidin. These results demonstrate whether the mAbs recognize similar or partially overlapping epitopes on IL-23.

One aspect of the method of the invention administers to a patient a pharmaceutical composition comprising

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gleatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(β-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating Crohn's disease, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluoroquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. Sec, e.g., Wells et al., eds., Pharmacotherapy Handbook, 2^{nd} Edition, Appleton and Lange, Stamford, CT (2000); PDR Pharmacopoeia, Tarascon Pocket Pharmacopoeia 2000, Deluxe Edition, Tarascon Publishing, Loma Linda, CA (2000); Nursing 2001 Handbook of Drugs, 21st edition, Springhouse Corp., Springhouse, PA, 2001; Health Professional's Drug Guide 2001, ed., Shannon, Wilson, Stang, Prentice-Hall, Inc, Upper Saddle River, NJ, each of which references are entirely incorporated herein by reference.

### Therapeutic Treatments

Typically, treatment of Crohn's disease is affected by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-23 specific antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of an anti-IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsich, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Example 1—Phase 2/3, Randomized, Double-Blind, Placebo- and Active-Controlled, Parallel-Group, Multicenter Protocol to Evaluate the Efficacy and Safety of Guselkumab in Participants with Moderately to Severely Active Crohn's Disease-Galaxi 2 and 3

### Preclinical Evidence Implicating IL-23 as a Target in Crohn's Disease

Genetic and animal model studies have explored the contribution of IL-12 and IL-23 in driving the pathophysiology of Crohn's disease. The results indicate that IL-23 plays a predominant role in inflammatory bowel disease (IBD) and emerging evidence suggests that blocking IL-23 alone may be a more effective strategy than blocking both IL-12 and IL-23.

Initial observations from genetic and animal model data suggest that Crohn's disease is mediated by IL-12 and/or IL-23, potentially through the Th1 and Th17 pathways they induce, respectively. However, increasing evidence suggests a predominant role for IL-23 in Crohn's disease. Genome-wide association studies identified polymorphisms in the IL-23R gene that are associated with Crohn's disease. The role of IL-23 in driving intestinal inflammation has been shown in several mouse models. Mice treated with anti-IL-23 antibodies exhibited attenuated inflammation, and mice with a genetic deletion of the p19 subunit of IL-23 are protected in several models of intestinal inflammation.

### Clinical Evidence Establishing Proof of Concept for Targeting IL-23 in Crohn's Disease

The potential therapeutic role of IL-23 in Crohn's disease was first established by clinical studies of IL-12/23p40 antagonists (briakinumab and ustekinumab). Ustekinumab (STELARA®) is approved for the treatment of moderately to severely active Crohn's disease. While these programs demonstrated that blockade of both IL-12 and IL-23 is effective in treating Crohn's disease, they could not ascertain the relative contributions of the 2 cytokines.

More recent studies of 2 anti-IL-23 antagonists, risankizumab (previously BI-655066) and brazikumab (formerly MEDI2070, AMG 139), and the Galaxi I study with guselkumab reported Phase 2 results demonstrating efficacy of IL-23 blockade in participants with moderately to severely active Crohn's disease. The magnitude of efficacy observed in each of these studies suggests the potential for improved efficacy compared with ustekinumab (anti-IL-12/23), recognizing the limitations of cross-study comparisons as well as the comparatively small size of the IL-23 Phase 2 studies.

Clinical Experience with IL-12/23-Targeted Therapy (Ustekinumab) in Crohn's Disease

The ustekinumab Phase 3 program in Crohn's disease included two 8-week studies evaluating the efficacy and safety of ustekinumab intravenous (IV) induction, and one maintenance study evaluating the efficacy and safety of ustekinumab subcutaneous (SC) maintenance, for a total duration of 52 weeks of treatment. Ustekinumab was evaluated in the full spectrum of biologic-eligible patients with Crohn's disease, ie, those who were conventional therapy failures and those who were biologic therapy failures. After a single ustekinumab ˜6 mg/kg IV induction dose at Week 0, approximately 21% and 40% of BIO-failure and CON-failure participants, respectively (versus approximately 7% and 20% of placebo-treated participants, respectively), achieved clinical remission at Week 8 (as evaluated by the Crohn's Disease Activity Index [CDAI]). Among participants who responded to ustekinumab IV induction and were rerandomized to receive ustekinumab SC maintenance 90 mg every 8 weeks (q8w) or 90 mg every 12 weeks (q12w), approximately 53% and 49% of participants were in clinical remission at Week 52, respectively, compared with 36% of participants who received placebo maintenance.

Clinical Experience with IL-23-Targeted Therapy in Crohn's Disease

Phase 2 studies of 2 IL-23 mAbs, risankizumab and brazikumab, demonstrated their efficacy in improving clinical signs and symptoms, reducing inflammatory biomarkers, and improving endoscopic findings in participants primarily with biologic-refractory Crohn's disease.

Cross-study comparisons of clinical remission rates with the IL-23 blockers suggest the potential for improved efficacy compared with ustekinumab. It is notable that the induction doses used in the studies of both risankizumab (200 and 600 mg IV at Weeks 0, 4, 8) and brazikumab (700 mg IV at Weeks 0, 4) were considerably higher than approved ustekinumab dosing (˜6 mg/kg IV at Week 0). A cross-compound meta-analysis suggests that the risankizumab dosing, in particular, may be at the higher end of the dose-response curve.

Furthermore, the Phase 2 study with risankizumab also suggested the potential that response rates may not reach maximum until after 6 months of treatment. With doses of 600 mg IV every 4 weeks (q4w) for up to 6 months, clinical remission rates of approximately 50% were observed in all-treated patients, substantially higher than remission rates previously reported for other agents, including ustekinumab, in similar study populations at similar follow-up time points. Of those participants who were in remission at 6 months and who continued risankizumab maintenance treatment (180 mg SC q8w), approximately 70% were in remission at 1 year. Ga

### Overall Rationale for Guselkumab in Crohn's Disease

In summary, the collective genetic and preclinical evidence implicates the prominent role of selectively targeting IL-23 in modulating the underlying pathophysiology of IBD. The available clinical experience of 2 IL-23 antagonists and the established evidence from an approved IL-12/23 antagonist (ustekinumab) have demonstrated proof of mechanism and proof of concept, respectively, for targeting IL-23 in the treatment of Crohn's disease. Together, the available evidence provide support for investigating guselkumab in the treatment of Crohn's disease.

### Objectives:

### Primary Objectives

To evaluate the clinical and endoscopic efficacy of guselkumab in participants with CD.
To evaluate the safety of guselkumab.

### Secondary Objectives

To evaluate the impact of guselkumab on health-related quality of life.
To evaluate the PK, immunogenicity, and PD of guselkumab therapy, including changes in CRP and fecal calprotectin.

### Endpoints:

There are 2 groups (Global and Region-specific) of co-primary and major secondary endpoints defined as follows:
Global endpoints for the United States and other countries/territories throughout the world as applicable.
Region-specific endpoints for other health authorities that preferred the original Week 12 co-primary endpoints.

### Global Endpoints

Global co-primary endpoints
   clinical response at Week 12 and clinical remission at Week 48
   clinical response at Week 12 and endoscopic response at Week 48
For these endpoints, comparisons were made between each guselkumab dose (guselkumab 200 mg IV q4w×3→100 mg SC q8w and guselkumab 200 mg IV q4w×3→200 mg SC q4w) and placebo.
Global major secondary endpoints
The Global major secondary endpoints are grouped into 3 categories based on evaluation timepoint and comparator; note that these are grouped for the purpose of categorization but are not presented in the order of testing in the multiplicity-controlled testing procedure.
The following Global major secondary endpoints evaluate the short-term efficacy of the combined guselkumab induction dose group (the 2 guselkumab treatment groups received the same 200 mg IV q4w×3 dosing regimen up to Week 12) versus placebo:
   clinical response at Week 4
   clinical remission at Week 12
   endoscopic response at Week 12
   fatigue response at Week 12
   clinical remission at Week 12 and endoscopic response at Week 12
   endoscopic remission (Global definition) at Week 12
The following Global long-term endpoints evaluate guselkumab versus placebo in each study:
   clinical response at Week 12 and 90-day corticosteroid-free clinical remission at Week 48
   clinical response at Week 12 and endoscopic remission (Global definition) at Week 48
The following Global long-term endpoints evaluate guselkumab versus ustekinumab:
   clinical remission at Week 48
   endoscopic response at Week 48
   clinical remission at Week 48 and endoscopic response at Week 48
   endoscopic remission (Global definition) at Week 48
   deep remission (Global definition) at Week 48

### Region-Specific Endpoints

Region-specific co-primary endpoints
   clinical remission at Week 12
   endoscopic response at Week 12
For these endpoints, comparisons were made between the combined guselkumab induction dose and group placebo.
Region-specific major secondary endpoints
The Region-specific major secondary endpoints are grouped into 3 categories based on evaluation timepoint and comparator; note that these are grouped for the purpose of categorization but are not presented in the order of testing in the multiplicity-controlled testing procedure.
The following Region-specific major secondary endpoints evaluate the short-term efficacy of guselkumab versus placebo:
   PRO-2 remission at Week 12
   fatigue response at Week 12
   endoscopic remission (Region-specific definition) at Week 12
The following Region-specific long-term endpoints evaluate guselkumab versus placebo:
   corticosteroid-free clinical remission at Week 48
   endoscopic response at Week 48
The following Region-specific long-term endpoints evaluate guselkumab versus ustekinumab:
   endoscopic remission (Region-specific definition) at Week 48
   clinical remission at Week 48
   endoscopic response at Week 48
   durable clinical remission at Week 48
   PRO-2 remission at Week 48
   clinical remission at Week 48 and endoscopic response at Week 48
   corticosteroid-free clinical remission at Week 48

### Definitions of Endpoints

The definitions associated with the endpoints are presented below. The endpoint definitions are common to both the Global and Region-specific endpoints unless noted otherwise.

| Endpoint | Definition |
|---|---|
| Clinical response | ≥100-point reduction from baseline in CDAI score or |
| | CDAI score <150. |
| Clinical remission | CDAI <150 |
| Endoscopic response | ≥50% improvement from baseline in the SES-CD or |
| | SES-CD score ≤2. |
| Corticosteroid-free clinical | CDAI score <150 at Week 48 and not receiving |
| remission at Week 48 | corticosteroids at Week 48 |
| Fatigue response | An improvement of ≥7 points in PROMIS Fatigue Short |
| | Form 7a |
| Durable clinical remission | CDAI <150 for ≥80% of all visits between Week 12 and |
| | Week 48 [ie, at least 8 of 10 visits]), which must include |
| | Week 48 |
| PRO-2 remission | Abdominal pain (AP) mean daily score at or below 1 |
| | [AP ≤1] and stool frequency (SF) mean daily score at or |
| | below 3 [SF ≤3], and no worsening of AP or SF from |
| | baseline. |
| Endoscopic remission (Global | SES-CD ≤4 with at least a 2-point reduction from |
| definition) | baseline and no subscore greater than 1 in any individual |
| | subcomponent. |
| Deep remission (Global definition) | Clinical remission and endoscopic remission (Global |
| | definition). |
| Endoscopic remission (Region- | SES-CD score ≤2 |
| specific definition) | |
| Deep remission (Region-specific | Clinical remission and endoscopic remission (Region- |
| definition) | specific definition). |

### Methodology:

CNTO1959CRD3001 is a randomized, double-blind, placebo and active-controlled (ustekinumab), parallel-group, multicenter study to evaluate the safety and efficacy of guselkumab in participants with moderately to severely active CD who have demonstrated an inadequate response to or failure to tolerate previous conventional therapy (i.e., oral corticosteroids or immunomodulators [6-MP/AZA/MTX]) or biologic therapy (i.e., TNF antagonists or vedolizumab).

Under the CNTO1959CRD3001 (GALAXI) protocol, there are 3 separate studies: a Phase 2 dose-ranging study (GALAXI 1) and 2 identical Phase 3 confirmatory studies (GALAXI 2 and GALAXI 3). The results of the GALAXI 2 and 3 studies through Week 48 are provided below.
At Week 0, participants were randomly allocated to GALAXI 2 or GALAXI 3 using a permuted block randomization with baseline CDAI score (≤300 or >300), baseline SES-CD score (≤12 or >12), prior BIO-Failure status (Yes/No), and baseline corticosteroid use (Yes/No) as the stratification variables.
Within each stratum, participants in GALAXI 2 and 3 were randomized in a 2:2:2:1 ratio to receive 1 of 2 dose regimens of guselkumab, ustekinumab, or placebo, respectively.
A minimum of 25% and a maximum of 50% of the total enrolled population were to be CON-Failure participants to ensure that the enrolled population was representative of the population of patients with moderately to severely active CD.
Allocation to treatment groups was performed using a central randomization center by means of an IWRS.

Participants were to remain on their assigned treatment regimens through the end of the 48-week study, except for the placebo treatment group as outlined below. No other dose adjustments were planned.
Group 1: Guselkumab Regimen 1 (200 mg IV q4w×3→200 mg SC q4w)
Participants received guselkumab 200 mg IV induction q4w from Week 0 through Week 8 (i.e., total of 3 IV doses). At Week 12, participants continued treatment with guselkumab 200 mg SC maintenance q4w through Week 44.
Group 2: Guselkumab Regimen 2 (200 mg IV q4w×3→100 mg SC q8w)
Participants received guselkumab 200 mg IV induction q4w from Week 0 through Week 8 (i.e., total of 3 IV doses). At Week 16, participants continued treatment with guselkumab 100 mg SC maintenance q8w through Week 40.
Group 3: Active Control-Ustekinumab (˜6 mg/kg IV→90 mg SC q8w)
Participants received a single ustekinumab IV induction dose at Week 0 (weight-based IV dose approximating 6 mg/kg as outlined below). At Week 8, participants received ustekinumab SC maintenance (90 mg SC q8w) through Week 40.
   Ustekinumab 260 mg (weight ≤55 kg)
   Ustekinumab 390 mg (weight >55 kg and ≤85 kg)
   Ustekinumab 520 mg (weight >85 kg)·
Group 4: Placebo→Placebo or Ustekinumab Crossover
Participants received placebo IV q4w from Week 0 through Week 8 (i.e., total of 3 IV doses). At Week 12, participants continued treatment based on their clinical response status as follows:
   Placebo responders: Continued placebo treatment from Week 12 through Week 44.
   Placebo nonresponders: Received a single ustekinumab IV induction dose at Week 12 (weight-based IV doses approximating 6 mg/kg as outlined above). At Week 20, participants received ustekinumab SC maintenance (90 mg SC q8w) through Week 44.

Clinical response was defined as a reduction from baseline (i.e., Week 0) in the CDAI score of ≥100 points or being in clinical remission (CDAI <150). To maintain the blind, participants in all treatment groups were assessed for their clinical response status at Week 12.

Efficacy, safety, PK, PD, immunogenicity, biomarkers, and medical resource utilization and health economics were assessed. A database lock (DBL) occurred after all randomized participants had either completed the Week 48 visit or had terminated study participation before Week 48. An independent DMC was established to monitor unblinded data on an ongoing basis to ensure the continuing safety of the participants enrolled in this study.

### Number of Participants (Planned and Analyzed):

Approximately 490 participants were planned for enrollment for each of Galaxi 2 and 3. A total of 524 participants were randomized of which 523 received at least 1 (partial or complete) dose of study intervention in Galaxi 2. Participant data were analyzed according to the assigned treatment.

### Power Calculations and Planned Sample Size:

The power for the key endpoints in the GALAXI 2 and 3 Phase 3 studies, as well as the number of participants necessary to assess the safety of guselkumab across the program, were considered in determining the appropriate sample size for these Phase 3 studies. Using the assumptions described in the table below, a sample size of approximately 490 participants (consisting of 140 participants in each of the two guselkumab dose groups, 140 participants in the ustekinumab group, and 70 participants in the placebo treatment group) provided at least 90% power for the co-primary endpoints (both global and region-specific) based on a chi-square test at the 0.05 (2-sided) significance level.

| | Placebo | Guselkumab |
|---|---|---|
| Endpoint | rate | rate |
| Clinical response at Week 12 and clinical | 10% | 50% |
| remission at Week 48 | | |
| Clinical response at Week 12 and endoscopic | 5% | 35% |
| response at Week 48 | | |
| Clinical remission at Week 12 | 15% | 50% |
| Endoscopic response at Week 12 | 13% | 33% |

### Statistical Considerations:

Primary Analysis sets for Efficacy and Safety: all randomized participants who received at least 1 (partial or complete) dose of study intervention and had a screening SES-CD score ≥6 [or ≥4 for participants with isolated ileal disease].

Intercurrent events (ICEs) are accounted for in the analysis of the efficacy endpoints. In particular, participants who had a prohibited change in concomitant CD medication, a CD-related surgery, or discontinued study agent due to lack of efficacy, an AE of worsening CD, or discontinuation of study agent for any reason other than COVID-19- or major disruption-related issues prior to the analysis timepoint were considered to not have met the endpoints (ie, composite strategy). However, participants who had discontinued study agent due to COVID-19 related reasons (excluding COVID-19 infection) or major disruption had their observed data used, if available (i.e., treatment policy strategy). An additional ICE of IWRS-determined clinical non-response for region-specific, long-term endpoints comparing guselkumab to placebo was also handled using the composite strategy.

Depending on the endpoint, the comparator will be either placebo or ustekinumab. As the 2 guselkumab dose groups received the same induction dose (200 mg IV), Week 12 comparisons between guselkumab and placebo will involve the combined guselkumab induction dose group. For endpoints during maintenance, each gueslkumab dose group (200 mg SC q4w and 100 mg SC q8w) will be compared to either placebo or ustekinumab depending on the endpoint.

The Common Risk Difference Test using Mantel-Haenszel stratum weights are used to compare each guselkumab dose group (or the combined guselkumab dose group) to placebo (or to ustekinumab) while adjusting for the stratification factors.

Missing data, after accounting for ICE strategies, occur in the absence of observed data when participants discontinue treatment due to COVID-19 related reasons (excluding COVID-19 infection) or major disruption, missed visits, or missed assessments. Participants with missing data for an endpoint are considered to be nonresponders (NRI) for that endpoint.

### Changes in Study Conduct

During the course of the GALAXI studies, changes to Phase 3 study endpoints and sample size were implemented and are summarized below. All changes were made prior to the completion of the Phase 3 Week 48 DBL, such that all Phase 3 data were blinded at the time of the changes. As noted in the GALAXI protocol, it was always intended that Phase 3 endpoints and assumptions were to be refined after analyzing the results of the Phase 2 study. The changes to the endpoints did not affect study design, eligibility criteria, data collection, or the overall study conduct of the GALAXI Phase 3 studies.

The original protocol of the Phase 3 studies had clinical remission at Week 12, assessed versus placebo, as the primary endpoint. However, included in the Phase 3 study hypothesis, was the intent to evaluate the relative performance of long-term treatment with guselkumab versus ustekinumab. For the major secondary hypotheses for comparison with ustekinumab, while the ultimate goal was to demonstrate that the efficacy of guselkumab is superior to ustekinumab, an initial test of non-inferiority was to be performed. The original sample size was based on clinical remission at Week 12 (compared to placebo) and clinical remission at Week 48 (compared to ustekinumab). Over time, with health authority feedback, the studies were modified to raise the importance of endoscopic outcomes, as reflected in the change to the co-primary endpoints: clinical remission at Week 12 and endoscopic response at Week 12 versus placebo (Protocol Amendment 3).

Further changes to endpoints and the study assumptions were made based on the results from the Phase 2 GALAXI 1 study, which was the first time that ustekinumab had been studied in a treat-through design. Tests for non-inferiority between ustekinumab and guselkumab were removed based on the difficulty of gaining agreement on a non-inferiority margin with health authorities. These changes are reflected in Protocol Amendment 5, which incorporated both a Global module and Region-specific module for endpoints and hypotheses, based on differing regional regulatory feedback. The Global module shifted to the following composite co-primary endpoints demonstrating short-term and long-term efficacy relative to placebo:
clinical response at Week 12 and clinical remission at Week 48
clinical response at Week 12 and endoscopic response at Week 48

The Region-specific module maintained the co-primary endpoints at Week 12 relative to placebo:
clinical remission at Week 12
endoscopic response at Week 12

Also added to the Region-specific module were 2 major secondary endpoints for long-term efficacy with a comparison to placebo:
corticosteroid-free clinical remission at Week 48
endoscopic response at Week 48

The Week 48 comparisons to ustekinumab were maintained as major secondary endpoints in both the Global and Region-specific module to evaluate long-term efficacy, with additional changes to the major secondary endpoints based on regulatory authorities' feedback. The composite endpoint of clinical remission at Week 48 and endoscopic response at Week 48 was added to both modules as a clinically relevant endpoint to measure both symptomatic and objective long-term improvement in an individual participant.

The sample size for the study was reduced at the time of Protocol Amendment 5, based on the Phase 2 assumptions and power calculations for all co-primary endpoints (Global and Region-specific) as well as the Week 48 major secondary endpoints with comparisons to placebo in the Region-specific module.

Changes in the conduct of the study that were implemented by 5 protocol amendments.

Two protocol amendments that were considered substantial occurred before the start of GALAXI 2.
Amendment 1 (10 Dec. 2018) included the following changes:
   Revision to the endoscopic inclusion criterion, which was changed to enroll study participants with evidence of ulceration in any 1 of the 5 ileocolonic segments.
   Additional clarifications throughout the protocol were based on feedback from health authorities, ethics committees, and investigative sites.
Amendment 2 (13 Nov. 2019) included the following changes:
   Specifying the guselkumab induction and maintenance doses to be administered in the Phase 3 studies (GALAXI 2 and GALAXI 3) based on the Week 12 IA from GALAXI 1.
   Clarifying information about the management of liver function test abnormalities. Of note, after the Week 12 IA to inform induction and maintenance dose selection for the Phase 3 studies, an urgent safety measure due to an event of toxic hepatitis in a participant in the guselkumab 1200 mg IV treatment group resulted in a temporary pause of induction dosing, effective 24 Oct. 2019.

Three protocol amendments that were considered substantial occurred after the start of GALAXI 2:
Amendment 3 (20 Oct. 2020) included the following changes:
   Added a real-life patient handling substudy to collect data about new 2 mL devices in the GALAXI 1 LTE.
   SES-CD inclusion criteria were revised from an SES-CD ≥3 to an SES-CD ≥6 (or ≥4 for participants with isolated ileal disease). Endoscopic response at Week 12 was added as a co-primary endpoint. These changes were made based on Health Authority feedback and Phase 2 (GALAXI 1) results.
Amendment 4 (21 Jul. 2021) included the following changes:
   Extended the duration of the LTE from 2 years to 4 years for all participants in the GALAXI studies.
   A Coronavirus Disease 2019 (COVID-19) Appendix was issued on 27 Apr. 2020 to provide guidance on study conduct during the COVID-19 pandemic.
Amendment 5 (12 Jul. 2022) included the following changes for the Phase 3 (GALAXI 2 and GALAXI 3) studies (the decision to reduce the sample size was not based on knowledge of any data from the Phase 3 GALAXI studies, as these studies were still blinded to the sponsor at the time of the amendment):
   Modification of the co-primary endpoints to have separate Global and Region-specific co-primary endpoints after Health Authority negotiations. Major secondary endpoints were also adjusted. Overall, the endpoints were changed to incorporate appropriate endpoints for the evaluation of short-term and long-term efficacy of guselkumab in a study design with placebo and active comparators.
   Decreased the sample size in the GALAXI Phase 3 studies from 1540 participants to approximately 980 participants (approximately 490 participants in each Phase 3 study). The reduced sample size provides adequate power for the Global and Region-specific co-primary endpoints, Region-specific major secondary endpoints versus placebo, and an adequate number of participants to assess the safety of guselkumab in CD.
   The Protocol was modified to include measures taken regarding not just the COVID 19 pandemic, but also any event of Major Disruption, including the COVID 19 pandemic, war, or natural disaster.

### Demography and Baseline Crohn's Disease Characteristics for GALAXI 2 and GALAXI 3 Pooled

Pooled analysis of GALAXI 2 and 3 indicated that both studies enrolled populations with similar demographics and disease severity.

### Prior and Concomitant Therapy

### Concomitant Therapy at Baseline

29.9% of participants were receiving immunomodulators (AZA, 6-MP, or MTX) (Table 1). The proportions were similar across treatment groups (Table 1).
37.4% of participants were receiving oral corticosteroids. The proportions were similar in the guselkumab 200 mg SC q4w treatment group (37.7%), guselkumab 100 mg SC q8w treatment group (37.8%), and ustekinumab treatment group (39.2%), but lower in the placebo treatment group (32.9%).

### Prior Therapy

52.8% of participants had a history of inadequate response or intolerance to previous biologic medication treatment (BIO-Failure group). Proportions were similar across treatment groups (ranging from 50.0% to 55.2%).
Approximately half of all participants (50.6%) had failed at least one TNF antagonist. Proportions were similar across treatment groups (ranging from 48.6% to 51.7%).
47.2% of participants had a history of inadequate response to conventional therapy but not biologic therapy (CON-Failure subpopulation). Proportions were similar across treatment groups (ranging from 44.8% to 50.0%).
41.9% of participants were naïve to biologic therapy (BIO-Naïve subpopulation). Proportions were similar across treatment groups (ranging from 40.6% to 44.7%). The BIO-Naïve subpopulation is a subset of the CON-Failure subpopulation.
0.8% of participants had previously used ustekinumab (1 participant each in the placebo treatment group and ustekinumab treatment group, and 2 participants in the guselkumab 200 mg SC q4w treatment group). Of these one had a prior failure to ustekinumab.
Data from the ATAS were consistent with data from the PAS.

All Global and Region-specific co-primary estimands, major secondary estimands, and subgroup analyses were based on the PAS. As a supportive analysis, each Global and Region-specific co-primary endpoint and all major secondary endpoints were also analyzed using the ATAS and the modified PAS. The participants included in the modified PAS were the same participants as in the PAS.

Participants in the PAS, ATAS, and modified PAS were analyzed according to their randomized study intervention, regardless of the study intervention they actually received.

For efficacy results through Week 12, the terminology used in-text for the treatment groups is as follows:

| Treatment Group | Terminology Used In-text |
|---|---|
| Combined guselkumab induction dose | Combined guselkumab 200 |
| group (guselkumab 200 mg IV q4w × 3)^{a} | mg IV |
| Placebo | Placebo |

^{a}As the 2 guselkumab treatment groups received the same 200 mg IV q4w × 3 dosing regimen up to Week 12, the 2 treatment groups are combined to evaluate the efficacy of guselkumab through Week 12.

For efficacy results through Week 48, the terminology used in-text for the treatment groups is as follows:

| | Terminology Used |
|---|---|
| Treatment Group | In-text |
| Guselkumab 200 mg IV q4w × 3 → 100 mg SC | Guselkumab 100 mg |
| q8w | SC q8w |
| Guselkumab 200 mg IV q4w × 3 → 200 mg SC | Guselkumab 200 mg |
| q4w | SC q4w |
| Combined guselkumab (ie, guselkumab 100 mg | Combined |
| SC q8w and 200 mg SC q4w) | guselkumab |
| Ustekinumab ~6 mg/kg IV → 90 mg SC q8w | Ustekinumab |
| Placebo | Placebo |

### Primary Analyses of Global Co-Primary Endpoints

Significantly greater proportions of participants in both guselkumab treatment groups achieved the composite co-primary endpoints of clinical response at Week 12 and clinical remission at Week 48, and clinical response at Week 12 and endoscopic response at Week 48 compared with the placebo treatment group (p-value <0.001 for all comparisons) (Tables 2 and 3).

### Clinical Remission.

As early as Week 4 (the first scheduled visit after the first dose of study intervention), a numerically greater proportion of participants in the combined guselkumab 200 mg IV treatment group (23.9%) achieved clinical remission compared with the placebo treatment group (13.2%). By Week 12, 47.1% of the combined guselkumab 200 mg IV treatment group achieved clinical remission compared with 22.4% of the placebo treatment group.
The proportions of participants who achieved clinical remission increased over time in the active treatment groups.
At all timepoints from Week 8 through Week 48, similar or numerically greater proportions of participants in the guselkumab 200 mg SC q4w and guselkumab 100 mg SC q8w treatment groups achieved clinical remission compared with the ustekinumab treatment group.

### Sustained Clinical Remission

Sustained clinical remission is defined as achieving clinical remission at both Week 12 and Week 48.
The proportions of participants in sustained clinical remission were numerically greater in the guselkumab 200 mg SC q4w treatment group (44.5%) and similar in the guselkumab 100 mg SC q8w treatment group (38.5%) compared with the ustekinumab treatment group (37.1%).

### Durable Clinical Remission

Durable clinical remission is defined as CDAI <150 for ≥80% of all visits between Week 12 and Week 48 (at least 8 of 10 visits), and must include Week 48.
The proportions of participants in durable clinical remission at Week 48 were numerically greater in the guselkumab 200 mg SC q4w treatment group (52.1%) and similar in the guselkumab 100 mg SC q8w treatment group (46.2%) compared with the ustekinumab treatment group (44.8%).

### Change in CDAI.

As early as Week 4 (the first scheduled visit after the first dose of study intervention) and through Week 12, participants in the combined guselkumab 200 mg IV treatment group achieved a greater mean change from baseline in CDAI scores (LS mean [SE]: −133.2 [5.54] at Week 12) compared with the placebo treatment group (LS mean [SE]: −40.5 [10.67] at Week 12).
At Week 48, the LS mean [SE] changes from baseline in CDAI scores were-193.0 [8.01] in the guselkumab 200 mg SC q4w treatment group, −163.0 [8.07] in the guselkumab 100 mg SC q8w treatment group, and −170.6 [8.07] in the ustekinumab treatment group.

### Week 48 Key Outcomes Among Participants Achieving Clinical Response at Week 12

Analyses were performed to compare the rates of clinical remission at Week 48 and endoscopic response at Week 48 between the subset of guselkumab participants achieving clinical response at Week 12 and the subset of placebo participants achieving clinical response at Week 12, respectively (Table 10). Among participants in the guselkumab treatment groups, the majority of participants who achieved clinical response at Week 12 went on to achieve clinical remission at Week 48 (>76%), and the majority of participants who achieved clinical response at Week 12 went on to achieve endoscopic response at Week 48 (>60%). Note that the number of placebo participants who were in clinical response at Week 12 was small, so caution is needed in interpreting the results in those participants.

### Week 48 Outcomes Among Participants not Achieving Clinical Response at Week 12

Analyses were performed to estimate the percentage of participants in the guselkumab and ustekinumab treatment groups without clinical response at Week 12 who achieved clinical remission at Week 48. The same analysis for nonresponders was performed for endoscopic response at Week 48 (Table 11).

Among participants in the guselkumab treatment groups without clinical response at Week 12:
The proportions of participants achieving clinical remission at Week 48 were 58.0% and 47.8% in the guselkumab 200 mg SC q4w and guselkumab 100 mg SC q8w treatment groups, respectively.
The proportions of participants achieving endoscopic response at Week 48 were 52.0% and 30.4% in the guselkumab 200 mg SC q4w and guselkumab 100 mg SC q8w treatment groups, respectively.

These outcomes support the benefit of continued treatment with guselkumab even if clinical response is not achieved at Week 12.

### Sustained Clinical Remission at Both Weeks 12 and 48

Sustaining clinical remission is an important goal in the management of CD. This analysis evaluated the number of participants in clinical remission at both Week 12 and Week 48.
Greater proportions of participants in the guselkumab treatment groups achieved sustained clinical remission compared with the placebo treatment group (Table 12).

### Tipping Point Analysis

Tipping point sensitivity analyses based on multiple imputation with Bernoulli draws were performed to determine the proportion of placebo participants who crossed over at Week 12 that would have needed to achieve clinical remission at Week 48 or endoscopic response at Week 48 to change the statistical significance of the comparison to guselkumab.

### Clinical Remission at Week 48

The tipping point analysis showed the following:
No significant difference in response of guselkumab 200 mg SC q4w treatment group over the placebo treatment group can be concluded when at least 75% of the 49 participants in the placebo treatment group who crossed over to ustekinumab were considered to have achieved clinical remission at Week 48.
No significant difference in response of guselkumab 100 mg SC q8w treatment group over the placebo treatment group can be concluded when at least 60% of the 49 participants in the placebo treatment group who crossed over to ustekinumab were considered to have achieved clinical remission at Week 48.

### Endoscopic Response at Week 48

The tipping point analysis showed the following:
No significant difference in response of guselkumab 200 mg SC q4w treatment group over the placebo treatment group can be concluded when at least 55% of the 49 participants in the placebo treatment group who crossed over to ustekinumab were considered to have achieved endoscopic response at Week 48.
No significant difference in response of guselkumab 100 mg SC q8w treatment group over the placebo treatment group can be concluded when at least 45% of the 49 participants in the placebo treatment group who crossed over to ustekinumab were considered to have achieved endoscopic response at Week 48.

### Results of Phase 3 GALAXI 2 and 3 Studies Through Week 48

### SUMMARY

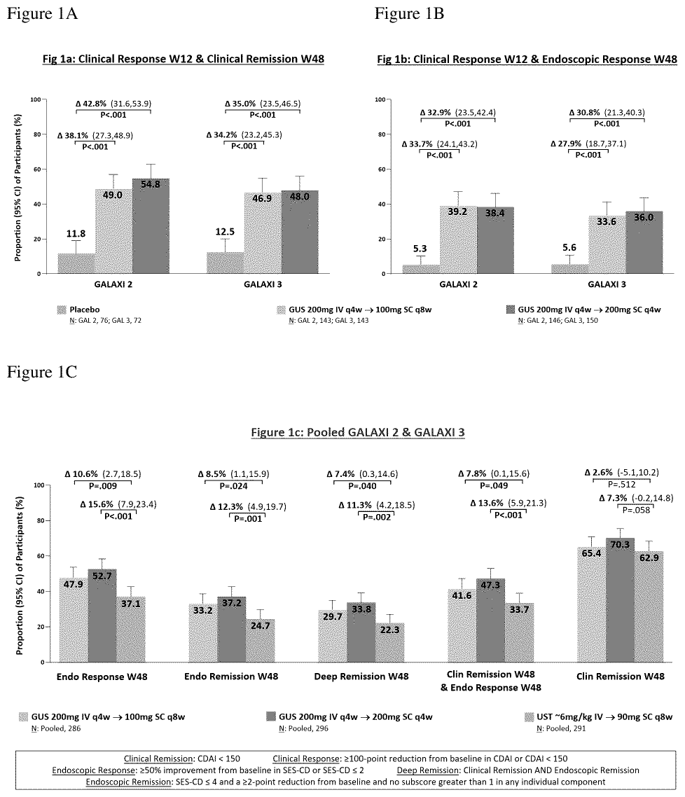

In both Galaxi 2 and 3 trials, significantly greater (P<0.001) proportions of patients receiving guselkumab 100 mg SC q8w or 200 mg SC q4w achieved the co-primary endpoints relative to placebo (as shown in FIGS. 1A and 1B). In the Week 48 pooled analyses (as shown in FIG. 1C) both guselkumab doses demonstrated superiority to ustekinumab for endoscopic response (GUS 100 mg SC q8w vs UST: Δ10.6%, GUS 200 mg SC q4w vs UST: Δ15.6%), endoscopic remission (Δ8.5%, Δ12.3%), deep remission (Δ7.4%, Δ11.3%), and clinical remission+endoscopic response (Δ7.8%, Δ13.6%).

### Embodiment List

The invention can be described with reference to the following numbered embodiments:

1. Use of an antibody to IL-23 for the treatment of moderate to severely active Crohn's disease in a patient, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, wherein the antibody is administered in an initial intravenous dose, an intravenous dose 4 weeks after initial treatment, an intravenous dose 8 weeks after initial treatment and a subcutaneous dose every 4 or 8 weeks after the dose at 8 weeks, and wherein the patient is a responder to the antibody as measured 12 or 48 weeks after the initial intravenous dose.

2. The use of embodiment 1, wherein the intravenous dose is selected from the group consisting of 1200 mg, 600 mg and 200 mg.

3. The use of embodiment 2, wherein the subcutaneous dose is 100 mg or 200 mg.

4. The use of embodiment 3, wherein the intravenous dose is 1200 mg and the subcutaneous dose is 200 mg every 4 weeks.

5. The use of embodiment 3, wherein the intravenous dose is 600 mg and the subcutaneous dose is 200 mg every 4 weeks.

6. The use of embodiment 3, wherein the intravenous dose is 600 mg and the subcutaneous dose is 100 mg every 8 weeks.

7. The use of embodiment 3, wherein the intravenous dose is 200 mg and the subcutaneous dose is 100 mg every 8 weeks.

8. The use of embodiment 3, wherein the intravenous dose is 200 mg and the subcutaneous dose is 200 mg every 4 weeks.

9. The use of embodiment 1, wherein the patient is identified as being in deep remission 48 weeks after the initial intravenous dose

10. The use of embodiment 1, wherein the patient is identified as meeting one or more clinical endpoints shown below:
(i) Change from Baseline in the Crohn's Disease Activity Index (CDAI) Score at Week 12;
(ii) Clinical remission at Week 12, defined as CDAI less than (<) 150 points;
(iii) Clinical response at Week 12, defined as greater than or equal to (>=) 100-point reduction from baseline in CDAI score or CDAI score <150;
(iv) Patient-Reported Outcome (PRO)-2 Remission at Week. 12 defined based on average daily stool frequency (SF) and average daily abdominal pain (AP) score;
(v) Clinical-Biomarker Response at Week 12 defined using clinical response based on the CDAI score and reduction from baseline in C-reactive protein (CRP) or fecal calprotectin;
(vi) Endoscopic Response at Week 12 measured by the Simple Endoscopic Score for Crohn's Disease (SES-CD);
(vii) Endoscopic Remission at Week 12 measured by the Simple Endoscopic Score for Crohn's Disease (SES-CD);
(viii) Clinical remission at Week 48 defined as CDAI score <150;
(ix) Durable Clinical Remission at Week 48 defined as CDAI <150 for most of all visits between Week 12 and Week 48;
(x) Corticosteroid-Free Clinical Remission at Week 48 defined as CDAI score <150 at Week 48 and not receiving corticosteroids at Week 48;
(xi) PRO-2 remission at Week 48 defined based on average daily stool frequency (SF) and average daily abdominal pain (AP) score;
(xii) Fatigue response at Week 12 based on the Patient-Reported Outcomes Measurement Information System (PROMIS); and
(xii) Endoscopic response at Week 48 measured by the Simple Endoscopic Score for Crohn's Disease (SES-CD).

11. The use of embodiment 10, wherein the clinical endpoint(s) is measured 4, 8, 12, 16, 20, 28, 32, 36, 40, 44 and/or 48 weeks after the initial intravenous dose.

12. The use of embodiment 1, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

13. The use of embodiment 1, further comprising administering to the patient one or more additional drugs used to treat Crohn's disease.

14. The use of embodiment 13, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

15. The use of embodiment 1, wherein the antibody comprises a light chain variable region amino acid sequence of SEQ ID NO: 8 and a heavy chain variable region amino acid sequence of SEQ ID NO: 7.

16. The use of embodiment 1, wherein the antibody comprises a light chain amino acid sequence of SEQ ID NO: 10 and a heavy chain amino acid sequence of SEQ ID NO: 9.

17. The use of embodiment 1, wherein the patient is considered a biologic therapy failure or intolerance for Crohn's disease (Bio-Failure).

18. The use of embodiment 1, wherein the patient is considered a conventional therapy failure or intolerance for Crohn's disease (Con-Failure).

19. The use of embodiment 1, wherein the subcutaneous does and frequency is adjust based on the patient failing to be in clinical response or clinical remission 12 weeks after the initial

TABLE 1
Summary of Concomitant Medications for Crohn's Disease at Baseline; Primary Analysis Set (CNTO1959CRD3001 GALAXI 2)

| | | | Guselkumab | | | | | | Ustekinumab ~6 | | | |
|---|---|---|---|---|---|---|---|---|---|---|---|---|
| | | | 200 mg IV | | 200 mg IV | | | | mg/kg IV → | | | |
| | | | q4w → 100 | | q4w → 200 | | | | 90 mg SC | | | |
| | Placebo^{a} | | mg SC q8w | | mg SC q4w | | Combined | | q8w | | Total | |
| Analysis set: Primary | 76 | | 143 | | 146 | | 289 | | 143 | | 508 | |
| Subjects with 1 or more concomitant | 47 | (61.8%) | 106 | (74.1%) | 108 | (74.0%) | 214 | (74.0%) | 109 | (76.2%) | 370 | (72.8%) |
| medications for Crohn's disease | | | | | | | | | | | | |
| Immunomodulatory drugs | 21 | (27.6%) | 43 | (30.1%) | 46 | (31.5%) | 89 | (30.8%) | 42 | (29.4%) | 152 | (29.9%) |
| 6-mercaptopurine/azathioprine | 18 | (23.7%) | 43 | (30.1%) | 45 | (30.8%) | 88 | (30.4%) | 39 | (27.3%) | 145 | (28.5%) |
| Methotrexate | 3 | (3.9%) | 0 | | 1 | (0.7%) | 1 | (0.3%) | 3 | (2.1%) | 7 | (1.4%) |
| Oral aminosalicylates | 23 | (30.3%) | 62 | (43.4%) | 61 | (41.8%) | 123 | (42.6%) | 64 | (44.8%) | 210 | (41.3%) |
| Antibiotics | 4 | (5.3%) | 6 | (4.2%) | 12 | (8.2%) | 18 | (6.2%) | 6 | (4.2%) | 28 | (5.5%) |
| Oral corticosteroid use | 25 | (32.9%) | 54 | (37.8%) | 55 | (37.7%) | 109 | (37.7%) | 56 | (39.2%) | 190 | (37.4%) |
| Corticosteroid use (excl. budesonide | 17 | (22.4%) | 39 | (27.3%) | 33 | (22.6%) | 72 | (24.9%) | 38 | (26.6%) | 127 | (25.0%) |
| and beclomethasone dipropionate) | | | | | | | | | | | | |
| Budesonide | 8 | (10.5%) | 17 | (11.9%) | 22 | (15.1%) | 39 | (13.5%) | 18 | (12.6%) | 65 | (12.8%) |
| Beclomethasone dipropionate | 0 | | 0 | | 0 | | 0 | | 0 | | 0 | |
| Corticosteroid dose, mg (excluding | | | | | | | | | | | | |
| budesonide and beclomethasone | | | | | | | | | | | | |
| diproprionate) | | | | | | | | | | | | |
| N | 17 | | 39 | | 33 | | 72 | | 38 | | 127 | |
| Mean (SD) | 18.2 | (8.65) | 17.5 | (9.64) | 18.7 | (9.47) | 18.1 | (9.51) | 17.1 | (8.27) | 17.8 | (8.99) |
| Median | 15.0 | | 20.0 | | 20.0 | | 20.0 | | 20.0 | | 20.0 | |
| Range | (10; 40) | | (3; 40) | | (5; 40) | | (3; 40) | | (5; 40) | | (3; 40) | |
| IQ range | (10.0; 20.0) | | (10.0; 20.0) | | (12.5; 20.0) | | (10.0; 20.0) | | (10.0; 20.0) | | (10.0; 20.0) | |
| Budesonide dose, mg | | | | | | | | | | | | |
| N | 8 | | 17 | | 22 | | 39 | | 18 | | 65 | |
| Mean (SD) | 7.5 | (2.27) | 8.5 | (1.59) | 7.6 | (2.29) | 8.0 | (2.04) | 8.3 | (1.28) | 8.0 | (1.88) |

| | | | | | | |
|---|---|---|---|---|---|---|
| Median | 9.0 | 9.0 | 9.0 | 9.0 | 9.0 | 9.0 |
| Range | (3; 9) | (3; 9) | (3; 9) | (3; 9) | (6; 9) | (3; 9) |
| IQ range | (6.0; 9.0) | (9.0; 9.0) | (6.0; 9.0) | (9.0; 9.0) | (9.0; 9.0) | (9.0; 9.0) |
| Beclomathasone dipropionate dose, mg | | | | | | |
| N | 0 | 0 | 0 | 0 | 0 | 0 |
| Mean (SD) | — | — | — | — | — | — |
| Median | — | — | — | — | — | — |
| Range | — | — | — | — | — | — |
| IQ range | — | — | — | — | — | — |

^{a}Placebo column includes all subjects randomized to placebo. At Week 12, subjects who were clinical responders continued placebo treatment and those who were nonresponders crossed over to ustekinumab.

TABLE 2
Summary of the Global Co-Primary Endpoints; Primary Analysis Set (CNTO1959CRD3001 GALAXI 2)

| | | | Guselkumab | | | |
|---|---|---|---|---|---|---|
| | | | 200 mg IV q4w → | | 200 mg IV q4w → | |
| | Placebo | | 100 mg SC q8w | | 200 mg SC q4w | |
| Analysis set: Primary | 76 | | 143 | | 146 | |
| Subjects in clinical response at Week 12 and clinical | 9 | (11.8%) | 70 | (49.0%) | 80 | (54.8%) |
| remission at Week 48^{a,b} | | | | | | |
| Adjusted treatment difference (95% CI)^{c} | | | 38.1 | (27.3, 48.9) | 42.8 | (31.6, 53.9) |
| p-value^{d} | | | <0.001 | | <0.001 | |
| Subjects in Clinical response at Week 12 and endoscopic | 4 | (5.3%) | 56 | (39.2%) | 56 | (38.4%) |
| response at Week 48^{a,b} | | | | | | |
| Adjusted treatment difference (95% CI)^{c} | | | 33.7 | (24.1, 43.2) | 32.9 | (23.5, 42.4) |

| | | |
|---|---|---|
| p-value^{d} | <0.001 | <0.001 |

Clinical response is defined as ≥100-point reduction from baseline in CDAI score or CDAI score <150.
Clinical remission is defined as CDAI score <150.
Endoscopic response is defined as ≥50% improvement from baseline in SES-CD score or SES-CD Score ≤2.
^{a}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{b}Missing data imputation: After accounting for ICE strategies, subjects who were missing CDAI score at Week 12 or CDAI or SES-CD score at Week 48 were considered not having achieved the endpoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 3
Summary of the Global Co-Primary Endpoints; Primary
Analysis Set (CNTO1959CRD3001 GALAXI 3)

| | | | Guselkumab | | | |
|---|---|---|---|---|---|---|
| | | | 200 mg IV q4w → | | 200 mg IV q4w → | |
| | Placebo | | 100 mg SC q8w | | 200 mg SC q4w | |
| Analysis set: Primary | 72 | | 143 | | 150 | |
| Subjects in clinical response at Week 12 and clinical | 9 | (12.5%) | 67 | (46.9%) | 72 | (48.0%) |
| remission at Week 48^{a,b} | | | | | | |
| Adjusted treatment difference (95% CI)^{c} | | | 34.2 | (23.2, 45.3) | 35.0 | (23.5, 46.5) |
| p-value^{d} | | | <0.001 | | <0.001 | |
| Subjects in Clinical response at Week 12 and | 4 | (5.6%) | 48 | (33.6%) | 54 | (36.0%) |
| endoscopic response at Week 48^{a,b} | | | | | | |
| Adjusted treatment difference (95% CI)^{c} | | | 27.9 | (18.7, 37.1) | 30.8 | (21.3, 40.3) |

| | | |
|---|---|---|
| p-value^{d} | <0.001 | <0.001 |

Clinical response is defined as ≥100-point reduction from baseline in CDAI score or CDAI score <150.
Clinical remission is defined as CDAI score <150.
Endoscopic response is defined as ≥50% improvement from baseline in SES-CD score or SES-CD Score ≤2.
^{a}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{b}Missing data imputation: After accounting for ICE strategies, subjects who were missing CDAI score at Week 12 or CDAI or SES-CD score at Week 48 were considered not having achieved the endpoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 4
Summary of the Global Major Secondary Endpoints With Short-term Placebo-based
Comparisons (Set 1); Primary Analysis Set (CNTO1959CRD3001 GALAXI 2)

| | | | Guselkumab | |
|---|---|---|---|---|
| | Placebo | | 200 mg IV Combined | |
| Analysis set: Primary | 76 | | 289 | |
| Week 12 | | | | |
| Subjects in clinical remission at Week 12^{a,b} | 17 | (22.4%) | 136 | (47.1%) |
| Adjusted treatment difference (95% CI)^{c} | | | 25.1 | (14.1, 36.2) |
| p-value^{d} | | | <0.001 | |
| Subjects in endoscopic response at Week 12^{a,b} | 8 | (10.5%) | 109 | (37.7%) |
| Adjusted treatment difference (95% CI)^{c} | | | 27.7 | (19.3, 36.1) |
| p-value^{d} | | | <0.001 | |
| Subjects in clinical remission at Week 12 and | 3 | (3.9%) | 62 | (21.5%) |
| endoscopic response at Week 12^{a,b} | | | | |
| Adjusted treatment difference (95% CI)^{c} | | | 17.6 | (11.1, 24.1) |
| p-value^{d} | | | <0.001 | |
| Subjects in fatigue response at Week 12^{a,b} | 22 | (28.9%) | 131 | (45.3%) |
| Adjusted treatment difference (95% CI)^{c} | | | 16.3 | (4.6, 28.1) |
| p-value^{d} | | | 0.006 | |
| Subjects in endoscopic remission at Week 12^{a,b} | 3 | (3.9%) | 64 | (22.1%) |
| Adjusted treatment difference (95% CI)^{c} | | | 18.4 | (11.7, 25.1) |
| p-value^{d} | | | <0.001 | |
| Week 4 | | | | |
| Subjects in clinical response at Week 4^{a,b} | 17 | (22.4%) | 114 | (39.4%) |
| Adjusted treatment difference (95% CI)^{c} | | | 16.3 | (5.4, 27.3) |

| | |
|---|---|
| p-value^{d} | 0.003 |

Clinical remission is defined as CDAI score <150.
Endoscopic response is defined as ≥50% improvement from baseline in SES-CD score or SES-CD Score ≤2.
Fatigue response is defined as improvement of ≥7 points in PROMIS Fatigue Short Form 7a.
Endoscopic remission (global definition) is defined as SES-CD Score ≤4 and at least a 2-point reduction from baseline and no subscore greater than 1 in any individual component.
Clinical response is defined as ≥100-point reduction from baseline in CDAI score or CDAI score <150.
^{a}ICE strategies: Subjects who had a CD-related surgery [ICE1], a prohibited change in concomitant CD medication [ICE2], or discontinued study agent due to lack of efficacy or an AE of worsening CD [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at the designated analysis timepoint.
^{b}Missing data imputation: After accounting for ICE strategies, subjects who were missing SES-CD or CDAI score or PROMIS Fatigue 7a score at the designated analysis timepoint were considered not having achieved the endpoint at that timepoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 5
Summary of the Global Major Secondary Endpoints With Long-term Placebo-based
Comparisons (Set 2); Primary Analysis Set (CNTO1959CRD3001 GALAXI 2)

| | | Guselkumab | | | |
|---|---|---|---|---|---|
| | | 200 mg IV q4w → | | 200 mg IV q4w → | |
| | Placebo | 100 mg SC q8w | | 200 mg SC q4w | |
| Analysis set: Primary | 76 | 143 | | 146 | |
| Subjects in Clinical response at Week 12 and 90-day | 7 (9.2%) | 67 | (46.9%) | 74 | (50.7%) |
| corticosteroid-free clinical remission at Week 48^{a,b} | | | | | |
| Adjusted treatment difference (95% CI)^{c} | | 38.7 | (28.4, 48.9) | 41.3 | (30.6, 52.0) |
| p-value^{d} | | <0.001 | | <0.001 | |
| Subjects in Clinical response at Week 12 and endoscopic | 2 (2.6%) | 38 | (26.6%) | 48 | (32.9%) |
| remission at Week 48^{a,b} | | | | | |
| Adjusted treatment difference (95% CI)^{c} | | 24.0 | (15.8, 32.2) | 30.0 | (21.4, 38.5) |

| | | |
|---|---|---|
| p-value^{d} | <0.001 | <0.001 |

Clinical response is defined as ≥100-point reduction from baseline in CDAI score or CDAI score <150.
Clinical remission is defined as CDAI score <150.
Endoscopic remission (global definition) is defined as SES-CD Score ≤4 and at least a 2-point reduction from baseline and no subscore greater than 1 in any individual component.
^{a}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{b}Missing data imputation: After accounting for ICE strategies, subjects who were missing CDAI score at Week 12 or CDAI or SES-CD score at Week 48 were considered not having achieved the endpoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 6
Summary of the Global Major Secondary Endpoints With Long-term Ustekinumab-
based Comparisons (Set 3); Primary Analysis Set(CNTO1959CRD3001 GALAXI 2)

| | Guselkumab | | | | Ustekinumab |
|---|---|---|---|---|---|
| | 200 mg IV q4w → | | 200 mg IV q4w → | | ~6 mg/kg IV → |
| | 100 mg SC q8w | | 200 mg SC q4w | | 90 mg SC q8w |
| Analysis set: Primary | 143 | | 146 | | 143 |
| Subjects in clinical remission at Week 48^{a,b} | 92 | (64.3%) | 109 | (74.7%) | 93 (65.0%) |
| Adjusted treatment difference (95% CI)^{c} | −0.6 | (−11.6, 10.5) | 9.5 | (−0.7, 19.8) | |
| p-value^{d} | 0.920 | | 0.069 | | |
| Subjects in endoscopic response at Week 48^{a,b} | 70 | (49.0%) | 82 | (56.2%) | 60 (42.0%) |
| Adjusted treatment difference (95% CI)^{c} | 7.0 | (−4.4, 18.3) | 14.5 | (3.6, 25.4) | |
| p-value^{d} | 0.230 | | 0.009 | | |
| Subjects in endoscopic remission at Week 48^{a,b} | 47 | (32.9%) | 65 | (44.5%) | 38 (26.6%) |
| Adjusted treatment difference (95% CI)^{c} | 6.4 | (−4.3, 17.1) | 17.9 | (7.3, 28.4) | |
| p-value^{d} | 0.239 | | <0.001 | | |
| Subjects in deep remission at Week 48^{a,b} | 43 | (30.1%) | 57 | (39.0%) | 37 (25.9%) |
| Adjusted treatment difference (95% CI)^{c} | 4.2 | (−6.2, 14.7) | 13.0 | (2.7, 23.4) | |
| p-value^{d} | 0.426 | | 0.014 | | |
| Subjects in clinical remission at Week 48 and | 60 | (42.0%) | 72 | (49.3%) | 56 (39.2%) |
| endoscopic response at Week 48^{a,b} | | | | | |
| Adjusted treatment difference (95% CI)^{c} | 2.8 | (−8.4, 14.1) | 10.2 | (−0.6, 21.0) | |

| | | |
|---|---|---|
| p-value^{d} | 0.620 | 0.065 |

Clinical remission is defined as CDAI score <150.
Endoscopic response is defined as ≥50% improvement from baseline in SES-CD score or SES-CD Score ≤2.
Endoscopic remission (global definition) is defined as SES-CD Score ≤4 and at least a 2-point reduction from baseline and no subscore greater than 1 in any individual component.
Deep remission is defined as achieving both clinical remission and endoscopic remission.
^{a}ICE strategies: Subjects who had a CD-related surgery [ICE1], a prohibited change in concomitant CD medication [ICE2], or discontinued study agent due to lack of efficacy, an AE of worsening CD or Week 20/24 non-responder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 48.
^{b}Missing data imputation: After accounting for ICE strategies, subjects who were missing CDAI or SES-CD at the designated analysis timepoint were considered not having achieved the endpoint at that timepoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 7
Summary of the Global Major Secondary Endpoints With Long-term Ustekinumab-based Comparisons
Pooled GALAXI 2 and 3; Primary Analysis Set (CNTO1959CRD3001 Pooled GALAXI 2 & 3)

| | Guselkumab | | | | Ustekinumab | |
|---|---|---|---|---|---|---|
| | 200 mg IV q4w → | | 200 mg IV q4w → | | ~6 mg/kg IV → | |
| | 100 mg SC q8w | | 200 mg SC q4w | | 90 mg SC q8w | |
| Analysis set: Pooled GALAXI 2 and 3 primary efficacy analysis set | 286 | | 296 | | 291 | |
| Subjects in clinical remission at Week 48^{a,b} | 187 | (65.4%) | 208 | (70.3%) | 183 | (62.9%) |
| Adjusted treatment difference (95% CI)^{c} | 2.6 | (−5.1, 10.2) | 7.3 | (-0.2, 14.8) | | |
| p-value^{d} | 0.512 | | 0.058 | | | |
| Subjects in endoscopic response at Week 48^{a,b} | 137 | (47.9%) | 156 | (52.7%) | 108 | (37.1%) |
| Adjusted treatment difference (95% CI)^{c} | 10.6 | (2.7, 18.5) | 15.6 | (7.9, 23.4) | | |
| p-value^{d} | 0.009 | | <0.001 | | | |
| Subjects in endoscopic remission at Week 48^{a,b} | 95 | (33.2%) | 110 | (37.2%) | 72 | (24.7%) |
| Adjusted treatment difference (95% CI)^{c} | 8.5 | (1.1, 15.9) | 12.3 | (4.9, 19.7) | | |
| p-value^{d} | 0.024 | | 0.001 | | | |
| Subjects in deep remission at Week 48^{a,b} | 85 | (29.7%) | 100 | (33.8%) | 65 | (22.3%) |
| Adjusted treatment difference (95% CI)^{c} | 7.4 | (0.3, 14.6) | 11.3 | (4.2, 18.5) | | |
| p-value^{d} | 0.040 | | 0.002 | | | |
| Subjects in clinical remission at Week 48 and endoscopic response | 119 | (41.6%) | 140 | (47.3%) | 98 | (33.7%) |
| at Week 48a^{a,b} | | | | | | |
| Adjusted treatment difference (95% CI)^{c} | 7.8 | (0.1, 15.6) | 13.6 | (5.9, 21.3) | | |

| | | |
|---|---|---|
| p-value^{d} | 0.049 | <0.001 |

Clinical remission is defined as CDAI score <150.
Endoscopic response is defined as ≥50% improvement from baseline in SES-CD score or SES-CD Score ≤2.
Endoscopic remission (global definition) is defined as SES-CD Score ≤4 and at least a 2-point reduction from baseline and no subscore greater than 1 in any individual component.
Deep remission is defined as achieving both clinical remission and endoscopic remission.
^{a}ICE strategies: Subjects who had a CD-related surgery [ICE1], a prohibited change in concomitant CD medication [ICE2], or discontinued study agent due to lack of efficacy, an AE of worsening CD or Week 20/24 non-responder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 48.
^{b}Missing data imputation: After accounting for ICE strategies, subjects who were missing CDAI or SES-CD score at the designated analysis timepoint were considered not having achieved the endpoint at that timepoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 8
Summary of the Global Major Secondary Endpoints With Short-
term Placebo-based Comparisons by Biologic Failure Status;
Primary Analysis Set (CNTO1959CRD3001 GALAXI 2)

| | | | Guselkumab |
|---|---|---|---|
| | Placebo | | 200 mg IV Combined |
| Analysis set: Primary | 76 | | 289 |
| Subgroup: BIO-Failure | 39 | | 150 |
| Clinical remission at Week | 9 | (23.1%) | 67 (44.7%) |
| 12^{a,b} | | | |
| Endoscopic response at Week | 2 | (5.1%) | 40 (26.7%) |
| 12^{a,b} | | | |
| Clinical remission and | 2 | (5.1%) | 22 (14.7%) |
| endoscopic response at | | | |
| Week 12^{a,b} | | | |
| Fatigue response at Week | 10 | (25.6%) | 62 (41.3%) |
| 12^{a,b} | | | |
| Endoscopic remission at | 2 | (5.1%) | 19 (12.7%) |
| Week 12^{a,b} | | | |
| Clinical response at Week 4^{a,b} | 8 | (20.5%) | 55 (36.7%) |
| Subgroup: CON-Failure | 37 | | 139 |
| Clinical remission at Week | 8 | (21.6%) | 69 (49.6%) |
| 12^{a,b} | | | |
| Endoscopic response at Week | 6 | (16.2%) | 69 (49.6%) |
| 12^{a,b} | | | |
| Clinical remission and | 1 | (2.7%) | 40 (28.8%) |
| endoscopic response at | | | |
| Week 12^{a,b} | | | |
| Fatigue response at Week | 12 | (32.4%) | 69 (49.6%) |
| 12^{a,b} | | | |
| Endoscopic remission at | 1 | (2.7%) | 45 (32.4%) |
| Week 12^{a,b} | | | |
| Clinical response at Week 4^{a,b} | 9 | (24.3%) | 59 (42.4%) |
| Subgroup: BIO-Naive | 34 | | 121 |
| Clinical remission at Week | 6 | (17.6%) | 60 (49.6%) |
| 12^{a,b} | | | |
| Endoscopic response at Week | 5 | (14.7%) | 62 (51.2%) |
| 12^{a,b} | | | |
| Clinical remission and | 0 | | 36 (29.8%) |
| endoscopic response at | | | |
| Week 12^{a,b} | | | |
| Fatigue response at Week | 11 | (32.4%) | 58 (47.9%) |
| 12^{a,b} | | | |
| Endoscopic remission at | 1 | (2.9%) | 39 (32.2%) |
| Week 12^{a,b} | | | |
| Clinical response at Week 4^{a,b} | 7 | (20.6%) | 52 (43.0%) |

Clinical remission is defined as CDAI score <150.
Endoscopic response is defined as ≥50% improvement from baseline in SES-CD score or SES-CD Score ≤2.
Fatigue response is defined as improvement of ≥7 points in PROMIS Fatigue Short Form 7a.
Endoscopic remission (global definition) is defined as SES-CD Score ≤4 and at least a 2-point reduction from baseline and no subscore greater than 1 in any individual component.
Clinical response is defined as ≥100-point reduction from baseline in CDAI score or CDAI score <150.
^{a}ICE strategies: Subjects who had a CD-related surgery [ICE1], a prohibited change in concomitant CD medication [ICE2], or discontinued study agent due to lack of efficacy or an AE of worsening CD [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12.
^{b}Missing data imputation: After accounting for ICE strategies, subjects who were missing CDAI, SES-CD or PROMIS Fatigue 7a score at the designated analysis timepoint were considered not having achieved the endpoint at that timepoint.

TABLE 9
Summary of the Global Co-Primary Endpoints and Major Secondary
Endpoints With Long-term Placebo-based Comparisons by Biologic
Failure Status; Primary Analysis Set (CNTO1959CRD3001 GALAXI 2)

| | | | Guselkumab | |
|---|---|---|---|---|
| | | | 200 mg IV q4w → | 200 mg IV q4w → |
| | Placebo | | 100 mg SC q8w | 200 mg SC q4w |
| Analysis set: Primary | 76 | | 143 | 146 |
| Subgroup: BIO-Failure | 39 | | 77 | 73 |
| Clinical response at Week 12 | 5 | (12.8%) | 30 (39.0%) | 38 (52.1%) |
| and clinical remission at | | | | |
| Week 48^{a,b} | | | | |
| Clinical response at Week 12 | 2 | (5.1%) | 28 (36.4%) | 19 (26.0%) |
| and endoscopic response at | | | | |
| Week 48^{a,b} | | | | |
| Clinical response at Week 12 | 4 | (10.3%) | 28 (36.4%) | 36 (49.3%) |
| and 90-day corticosteroid- | | | | |
| free clinical remission at | | | | |
| Week 48^{a,b} | | | | |
| Clinical response at Week 12 | 1 | (2.6%) | 19 (24.7%) | 15 (20.5%) |
| and endoscopic remission at | | | | |
| Week 48^{a,b} | | | | |
| Subgroup: CON-Failure | 37 | | 66 | 73 |
| Clinical response at Week 12 | 4 | (10.8%) | 40 (60.6%) | 42 (57.5%) |
| and clinical remission at | | | | |
| Week 48^{a,b} | | | | |
| Clinical response at Week 12 | 2 | (5.4%) | 28 (42.4%) | 37 (50.7%) |
| and endoscopic response at | | | | |
| Week 48^{a,b} | | | | |
| Clinical response at Week 12 | 3 | (8.1%) | 39 (59.1%) | 38 (52.1%) |
| and 90-day corticosteroid- | | | | |
| free clinical remission at | | | | |
| Week 48^{a,b} | | | | |
| Clinical response at Week 12 | 1 | (2.7%) | 19 (28.8%) | 33 (45.2%) |
| and endoscopic remission at | | | | |
| Week 48^{a,b} | | | | |
| Subgroup: BIO-Naive | 34 | | 58 | 63 |
| Clinical response at Week 12 | 3 | (8.8%) | 35 (60.3%) | 37 (58.7%) |
| and clinical remission at | | | | |
| Week 48^{a,b} | | | | |
| Clinical response at Week 12 | 2 | (5.9%) | 26 (44.8%) | 31 (49.2%) |
| and endoscopic response at | | | | |
| Week 48^{a,b} | | | | |
| Clinical response at Week 12 | 2 | (5.9%) | 34 (58.6%) | 33 (52.4%) |
| and 90-day corticosteroid- | | | | |
| free clinical remission at | | | | |
| Week 48^{a,b} | | | | |
| Clinical response at Week 12 | 1 | (2.9%) | 19 (32.8%) | 27 (42.9%) |
| and endoscopic remission at | | | | |
| Week 48^{a,b} | | | | |

Clinical response is defined as ≥100-point reduction from baseline in CDAI score or CDAI score <150.
Clinical remission is defined as CDAI score <150.
Endoscopic response is defined as ≥50% improvement from baseline in SES-CD score or SES-CD Score ≤2.
Endoscopic remission (global definition) is defined as SES-CD Score ≤4 and at least a 2-point reduction from baseline and no subscore greater than 1 in any individual component.
^{a}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{b}Missing data imputation: After accounting for ICE strategies, subjects who were missing CDAI score at Week 12 or CDAI or SES-CD score at Week 48 were considered not having achieved the endpoint.

TABLE 10
Summary of the Durability of Efficacy with Placebo-based Comparisons (Supportive to Estimands 1G and 2G) (Placebo
Crossovers to Ustekinumab are Considered Intercurrent Event); Primary Analysis Set (CNTO1959CRD3001 GALAXI 2)

| | | | Guselkumab | | | | | | Ustekinumab | |
|---|---|---|---|---|---|---|---|---|---|---|
| | | | 200 mg IV q4w → | | 200 mg IV q4w → | | | | ~6 mg/kg IV → | |
| | Placebo | | 100 mg SC q8w | | 200 mg SC q4w | | Combined | | 90 mg SC q8w | |
| Analysis set: Primary | 76 | | 143 | | 146 | | 289 | | 143 | |
| Subset: Subjects achieving clinical response at | 22 | (28.9%) | 91 | (63.6%) | 93 | (63.7%) | 184 | (63.7%) | 81 | (56.6%) |
| Week 12^{a,b} | | | | | | | | | | |
| Subjects in clinical remission at Week 48^{a,b} | 9 | (40.9%) | 70 | (76.9%) | 80 | (86.0%) | 150 | (81.5%) | 66 | (81.5%) |
| Adjusted treatment difference (95% CI)^{c} | | | 36.7 | (15.2, 58.2) | 45.3 | (23.4, 67.1) | 40.6 | (19.8, 61.3) | 39.3 | (16.7, 61.9) |
| p-value^{d} | | | <0.001 | | <0.001 | | <0.001 | | <0.001 | |
| Subjects in endoscopic response at Week 48^{a,b} | 4 | (18.2%) | 56 | (61.5%) | 56 | (60.2%) | 112 | (60.9%) | 43 | (53.1%) |
| Adjusted treatment difference (95% CI)^{c} | | | 44.3 | (25.0, 63.6) | 44.2 | (25.3, 63.2) | 43.6 | (26.1, 61.0) | 29.9 | (9.0, 50.8) |

| | | | | |
|---|---|---|---|---|
| p-value^{d} | <0.001 | <0.001 | <0.001 | 0.005 |

Clinical response is defined as ≥100-point reduction from baseline in CDAI score or CDAI score <150.
Clinical remission is defined as CDAI score <150.
Endoscopic response is defined as ≥50% improvement from baseline in SES-CD score or SES-CD Score ≤2.
^{a}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{b}Missing data imputation: After accounting for ICE strategies, subjects who were missing CDAI score at Week 12 or CDAI or SES-CD score at Week 48 were considered not having achieved the endpoint.
^{c}The adjusted treatment difference and the CIs were based on the common risk difference by use of Mantel-Haenszel stratum weights and the Sato variance estimator.
^{d}The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

TABLE 11
Summary of the Durability of Efficacy with Ustekinumab-based Comparisons (Supportive to
Estimands 1G and 2G); Week 12 Treated Primary Analysis Set (CNTO1959CRD3001 GALAXI 2)

| | Guselkumab | | | Ustekinumab |
|---|---|---|---|---|
| | 200 mg IV q4w → | 200 mg IV q4w → | | ~6 mg/kg IV → |
| | 100 mg SC q8w | 200 mg SC q4w | Combined | 90 mg SC q8w |
| Analysis set: Week 12 Treated Primary^{a} | 137 | 143 | 280 | 133 |
| Subset: Subjects not achieving clinical response | 46 (33.6%) | 50 (35.0%) | 96 (34.3%) | 52 (39.1%) |
| at Week 12^{b} | | | | |
| Subjects in clinical remission at Week 48^{c,d} | 22 (47.8%) | 29 (58.0%) | 51 (53.1%) | 27 (51.9%) |
| 95% CI^{e} | (33.4, 62.3) | (44.3, 71.7) | (43.1, 63.1) | (38.3, 65.5) |
| Subjects in endoscopic response at Week 48^{c,d} | 14 (30.4%) | 26 (52.0%) | 40 (41.7%) | 17 (32.7%) |
| 95% CI^{e} | (17.1, 43.7) | (38.2, 65.8) | (31.8, 51.5) | (19.9, 45.4) |

Clinical response is defined as ≥100-point reduction from baseline in CDAI score or CDAI score <150.
Clinical remission is defined as CDAI score <150.
Endoscopic response is defined as ≥50% improvement from baseline in SES-CD score or SES-CD Score ≤2.
^{a}Week 12 treated primary analysis set includes subjects in the primary analysis set who were treated at Week 12 and did not have ICE 1-3 or 5 prior to Week 12.
^{b}Missing data imputation: Subjects without ICEs before Week 12 and who were missing CDAI score at Week 12 were considered not having achieved clinical response at Week 12.
^{c}ICE strategies for ICEs occuring after Week 12: Subjects who had a CD-related surgery [ICE1], a prohibited change in concomitant CD medication [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 48.
^{d}Missing data imputation: After accounting for ICE strategies for ICEs occuring after Week 12, subjects who were missing CDAI or SES-CD score at Week 48 were considered not having achieved the endpoint.
^{e}The confidence intervals for the proportion of subjects meeting the endpoint in each treatment group were based on the normal approximation confidence limits. In cases of rare events, the exact confidence limits were provided.

TABLE 12
Supportive Analysis for Global Co-Primary Endpoint
(Supportive to Estimand 1G): Number of Subjects
in Clinical Remission at Both Weeks 12 and 48;
Primary Analysis Set (CNTO1959CRD3001 GALAXI 2)

| | Placebo |
|---|---|
| Analysis set: Primary | 76 |
| Subjects in clinical remission at | 8 (10.5%) |
| both Weeks 12 and 48^{a,b,c} | |
| 95% CI^{d} | (3.6, 17.4) |
| Adjusted treatment difference | |
| (95% CI)^{e} | |
| p-value ^{f} | |

Key: ICE = intercurrent event
^{a}Clinical remission is defined as CDAI score <150.
^{b}ICE strategies: Subjects who had a CD-related surgery [ICE1], a prohibited change in concomitant CD medication (including placebo subjects in clinical response who cross over to ustekinumab at Week 12) [ICE2], or discontinued study agent due to lack of efficacy, an AE of worsening CD or Week 20/24 nonresponder [ICE3] or discontinued study agent for any other reason other than COVID-19 related reasons or regional crisis [ICE5] prior to the analysis timepoint were considered not to have met the endpoint criteria. Subjects who had discontinued study agent due to COVID-19 related reasons (excluding COVID-19 infection) or regional crisis [ICE4] had their observed data used, if available, to determine responder and nonresponder status at Week 12 and Week 48.
^{c}Missing data imputation: After accounting for ICE strategies, subjects who were missing CDAI score at Week 12 or Week 48 were considered not having achieved the endpoint at Week 12 or Week 48.
^{d}The confidence intervals for the proportion of subjects meeting the endpoint in each treatment group were based on the normal approximation confidence limits. In cases of rare events, the exact confidence limits were provided.
^{e}The adjusted treatment difference and the CIs were based on the common risk difference by use of Mantel-Haenszel stratum weights and the Sato variance estimator.
^{f} The p-values were based on the common risk difference by use of Mantel-Haenszel stratum weights and the Sato variance estimator. The stratification variables used are baseline CDAI score (≤300 or >300), baseline SES-CD score (≤12 or >12), BIO-Failure status (Yes or No), and baseline corticosteroid use (Yes or No).

What is claimed is:

1. A method of treating moderate to severely active Crohn's disease in a patient, comprising administering an antibody to IL-23 to the patient, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, wherein the antibody is administered in an initial intravenous dose, an intravenous dose 4 weeks after initial treatment, an intravenous dose 8 weeks after initial treatment and a subcutaneous dose every 4 or 8 weeks after the dose at 8 weeks, and wherein the patient is a responder to the antibody as measured 12 or 48 weeks after the initial intravenous dose.

2. The method of claim 1, wherein the intravenous dose is selected from the group consisting of 1200 mg, 600 mg and 200 mg.

3. The method of claim 2, wherein the subcutaneous dose is 100 mg or 200 mg.

4. The method of claim 3, wherein the intravenous dose is 1200 mg and the subcutaneous dose is 200 mg every 4 weeks.

5. The method of claim 3, wherein the intravenous dose is 600 mg and the subcutaneous dose is 200 mg every 4 weeks.

6. The method of claim 3, wherein the intravenous dose is 600 mg and the subcutaneous dose is 100 mg every 8 weeks.

7. The method of claim 3, wherein the intravenous dose is 200 mg and the subcutaneous dose is 100 mg every 8 weeks.

8. The method of claim 3, wherein the intravenous dose is 200 mg and the subcutaneous dose is 200 mg every 4 weeks.

9. The method of claim 1, wherein the patient is identified as being in deep remission 48 weeks after the initial intravenous dose.

10. The method of claim 1, wherein the patient is identified as meeting one or more clinical endpoints shown below:
(i) Change from Baseline in the Crohn's Disease Activity Index (CDAI) Score at Week 12;
(ii) Clinical remission at Week 12, defined as CDAI less than (<) 150 points;
(iii) Clinical response at Week 12, defined as greater than or equal to (>=) 100-point reduction from baseline in CDAI score or CDAI score <150;
(iv) Patient Reported Outcome (PRO)-2 Remission at Week 12 defined based on average daily stool frequency (SF) and average daily abdominal pain (AP) score;
(v) Clinical-Biomarker Response at Week 12 defined using clinical response based on the CDAI score and reduction from baseline in C-reactive protein (CRP) or fecal calprotectin;
(vi) Endoscopic Response at Week 12 measured by the Simple Endoscopic Score for Crohn's Disease (SES-CD);
(vii) Endoscopic Remission at Week 12 measured by the Simple Endoscopic Score for Crohn's Disease (SES-CD);
(viii) Clinical remission at Week 48 defined as CDAI score <150;
(ix) Durable Clinical Remission at Week 48 defined as CDAI <150 for most of all visits between Week 12 and Week 48;
(x) Corticosteroid-Free Clinical Remission at Week 48 defined as CDAI score <150 at Week 48 and not receiving corticosteroids at Week 48;
(xi) PRO-2 remission at Week 48 defined based on average daily stool frequency (SF) and average daily abdominal pain (AP) score;
(xii) Fatigue response at Week 12 based on the Patient-Reported Outcomes Measurement Information System (PROMIS); and
(xiii) Endoscopic response at Week 48 measured by the Simple Endoscopic Score for Crohn's Disease (SES-CD).

11. The method of claim 10, wherein the clinical endpoint(s) is measured 4, 8, 12, 16, 20, 28, 32, 36, 40, 44 and/or 48 weeks after the initial intravenous dose.

12. The method of claim 1, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

13. The method of claim 1, further comprising administering to the patient one or more additional drugs used to treat Crohn's disease.

14. The method of claim 13, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

15. The method of claim 1, wherein the antibody comprises a light chain variable region amino acid sequence of SEQ ID NO: 8 and a heavy chain variable region amino acid sequence of SEQ ID NO: 7.

16. The method of claim 1, wherein the antibody comprises a light chain amino acid sequence of SEQ ID NO: 10 and a heavy chain amino acid sequence of SEQ ID NO: 9.

17. The method of claim 1, wherein the patient is considered a biologic therapy failure or intolerance for Crohn's disease (Bio-Failure).

18. The method of claim 1, wherein the patient is considered a conventional therapy failure or intolerance for Crohn's disease (Con-Failure).

19. The method of claim 1, wherein the subcutaneous does and frequency is adjust based on the patient failing to be in clinical response or clinical remission 12 weeks after the initial intravenous dose.


SEQUENCE LISTING (10 sequences)

SEQ ID NO: 1 - protein, 5 aa, Homo sapiens
NYWIG

SEQ ID NO: 2 - protein, 17 aa, Homo sapiens
IIDPSNSYTRYSPSFQG

SEQ ID NO: 3 - protein, 8 aa, Homo sapiens
WYYKPFDV

SEQ ID NO: 4 - protein, 14 aa, Homo sapiens
TGSSSNIGSGYDVH

SEQ ID NO: 5 - protein, 7 aa, Homo sapiens
GNSKRPS

SEQ ID NO: 6 - protein, 11 aa, Homo sapiens
ASWTDGLSLVV

SEQ ID NO: 7 - protein, 117 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS